(19) United States
(12) Patent Application Publication
(10) Pub. No.: US 2024/0367112 A1
(43) Pub. Date: Nov. 7, 2024

(54) AMPHIPHILIC POLYAMPHOLYTES AND RELATED MEMBRANES

(71) Applicant: Trustees of Tufts College, Medford, MA (US)

(72) Inventors: Luca Mazzaferro, Somerville, MA (US); Ayse Asatekin Alexiou, Arlington, MA (US)

(21) Appl. No.: 18/684,986

(22) PCT Filed: Aug. 22, 2022
(86) PCT No.: PCT/US2022/041055

§ 371 Date: Feb. 20, 2024

Related U.S. Application Data
(60) Provisional application No. 63/235,454, filed on Aug. 20, 2021.

(51) Int. Cl. B01D 69/02; B01D 69/10; B01D 71/40; B01D 71/76; C02F 1/44; C02F 101/30; C07K 1/34

(52) U.S. Cl. CPC B01D 69/02; B01D 69/10; B01D 71/401; B01D 71/76; C02F 1/444; C07K 1/34; B01D 2325/02831; B01D 2325/02832; B01D 2325/04; B01D 2325/14; B01D 2325/16; C02F 2101/30

(57) ABSTRACT

Disclosed herein are a series of terpolymers, comprising hydrophobic, anionic, and cationic repeat units; and their use in anti-fouling coatings and highly fouling-resistant membrane filters whose selectivity can be controlled through the modulation of the polymer composition. Also provided are methods of purifying compositions such as peptides and proteins, or methods of decontaminating or removing an organic solute from water using the membrane filters of the invention.

### RELATED APPLICATION

This application claims the benefit of priority to U.S. Provisional Patent Application No. 63/235,454, filed Aug. 20, 2021.

### GOVERNMENT SUPPORT

This invention was made with government support under grant DE-FE0031851 awarded by the United States Department of Energy, and grants 1553661 and 1904465 awarded by the National Science Foundation. The government has certain rights in the invention.

### BACKGROUND

Fouling, or the deposition of unwanted material on surfaces, is a severe problem in many applications where surfaces are in contact with seawater or surface water. Fouling phenomena affect ship hulls and other surfaces exposed to the marine environment,¹⁻⁴ pipes and other equipment used in deep water oil extraction,⁵⁻⁹ piping and heat transfer components,¹⁰⁻¹² and water filtration membranes.¹³⁻¹⁷ Each year, fouling causes significant economic impact through decreased productivity, increased energy use, and frequent maintenance and downtime for affected equipment. Therefore, there is a need for materials and coatings that resist fouling when exposed to various aqueous solutions containing biomacromolecules, oil, and microorganisms.

Fouling is a particularly impactful obstacle to the reliable use of membranes for various applications, particularly those where feeds have high organic content (e.g., water and wastewater treatment, food and beverage industry, and bioseparations). Severe declines in membrane permeability and changes in membrane selectivity due to fouling are common. Fouling management is a significant component of costs associated with membrane systems, requiring increased energy use, regular cleanings involving downtime, maintenance and chemical use, and more complex processes.

In addition, membranes with improved selectivity, or ability to separate solutes with better precision, offer to improve the economic feasibility and energy efficiency of membrane processes for many applications. For instance, membranes with tunable pore size and charge at the nanometer scale would be useful in bioseparations, treating complex wastewaters, selective removal of organics (e.g., dyes) and other contaminants from electrolyte solutions for reuse, water softening, pretreatment of seawater for desalination by reverse osmosis (RO), and final treatment of secondary and tertiary wastewater effluents. In all these cases, it is crucial for selectivity to be coupled with fouling resistance.

Researchers have screened a broad range of surface chemistries to identify fouling-resistant functional groups and gain an understanding of the mechanism of fouling resistance. They observed that surfaces resistant to protein adsorption share four molecular-level characteristics: they are hydrophilic, include hydrogen bond acceptors but no hydrogen bond donors, and are neutral in charge.¹⁸⁻¹⁹ Several promising chemistries, including PEO brushes,¹⁸⁻¹⁹ mannitol groups,²⁰ chimeric peptoids,^{21, 22} zwitterions,^{18, 23, 24} and polyampholytes^{25, 26} share these characteristics.

Of these, zwitterions have received extensive attention especially in the membrane field due to their facile synthesis and incorporation into existing membranes, often by post-processing methods like grafting.^{27, 28} The performance of zwitterions is comparable to the best known systems for resisting protein adsorption^{18, 23, 24, 29, 30} and cell adhesion.^{23, 24} Their biocompatibility and fouling resistance is attributed to the very small perturbation effect at the polymer-water interface.^{29, 31}

While zwitterions have anionic and cationic groups in the same monomer unit, polyampholytes have anionic and cationic groups in different monomer units. Similar to zwitterions, polyampholytes can exhibit excellent fouling resistance^{25, 26}. As previously stated, while zwitterions have been extensively studied in membrane systems, this is not true for polyampholytes. The few examples where polyampholytes are used in membrane systems mostly involve surface modification of common ultrafiltration membranes by graft copolymerization (UV light or redox initiated) of anionic and cationic monomer units to improve fouling resistance^{26, 32}.

Previous research has demonstrated that random zwitterionic amphiphilic copolymer (r-ZAC) membranes can be formed via the self-assembly of zwitterionic nanodomains acting as a network of effective nanochannels for water permeation³³. The main characteristics of r-ZAC membranes include excellent fouling resistance, a ˜1-1.5 nm size cutoff, and low salt ion retention^{33, 34}. However, while various zwitterionic monomers can be synthesized, only a handful are commercially available, some from only one supplier. Moreover, most zwitterionic monomers exhibit poor solubility in many solvents, further adding to their synthesis challenges. As a result, the range of r-ZAC chemistries that can feasibly be converted to membranes produced at a large scale are limited. Ionic or ionizable monomers, on the other hand, are widely available, cheaper, and typically easier to solubilize and synthesize.

Thus, there exists a need to develop fouling-resistant polymeric materials that may be synthesized from readily-available ionic or ionizable monomers and that may be employed in anti-fouling coatings and tunable membranes useful for separation applications.

### SUMMARY

In some aspects, the invention provides polymers, comprising:
(a) a plurality of hydrophobic repeat units;
(b) a plurality of a cationic repeat units; and
(c) a plurality of an anionic repeat units;
wherein the hydrophobic repeat units, cationic repeat units, and anionic repeat units are in random or statistical order.

In other aspects, the invention provides thin film composite membranes, comprising a porous substrate, and a selective layer comprising a polymer of the invention, wherein an average effective pore size of the porous substrate is larger than an average effective pore size of the selective layer; and the selective layer is disposed on a surface of the porous substrate.

In other aspects, the invention provides materials with an anti-fouling coating, comprising a solid substrate, and a layer comprising a polymer of the invention, wherein the layer is disposed on a surface of the solid substrate.

In further aspects, the invention provides methods of purifying a peptide or protein, comprising contacting at a first flow rate the thin film composite membrane of the invention with a mixture comprising one or more peptides or proteins, whereby a portion of the mixture is retained by the thin film composite membrane.

The invention also provides methods of removing an organic solute from water, comprising contacting at a first flow rate the thin film composite membrane of the invention with an aqueous solution comprising an organic solute, whereby the organic solute is retained by the thin film composite membrane.

The invention also provides methods of decontaminating water, comprising contacting at a first flow rate the thin film composite membrane of the invention with a mixture comprising water and one or more contaminants, whereby one or more contaminants are retained by the thin film composite membrane.

In other aspects, the invention provides methods of size-selective separation, comprising contacting at a first flow rate the thin film composite membrane of the invention with a mixture comprising one or more particles of differing sizes, whereby one or more particles of a particular size are retained by the thin film composite membrane.

### BRIEF DESCRIPTION OF THE DRAWINGS

FIG. 1 shows the NMR spectrum of PTMS chemical structure indicating copolymerization of the three monomer units.

FIG. 2A shows a schematic of casting PTMS on a PS35 base membrane.

FIG. 2B shows SEM images of PTMS coated on top of PS35 (left) and uncoated PS35 (right). The selective layer is visible on the left.

FIG. 3 shows the NMR spectrum of PTMS− chemical structure indicating copolymerization of the three monomer units.

FIG. 4 shows SEM images of PTMS− coated on top of PS35 (left) and uncoated PS35 (right). The selective layer is visible on the left.

FIG. 5 shows the NMR spectrum of PTMS+ chemical structure indicating copolymerization of the three monomer units.

FIG. 6 shows SEM images of PTMS+ coated on top of PS35 (left) and uncoated PS35 (right). The selective layer is visible on the left.

FIG. 7 shows the NMR spectrum of PTMM chemical structure indicating copolymerization of the three monomer units.

FIG. 8 shows SEM images of PTMM coated on top of PS35 (left) and uncoated PS35 (right). The selective layer is visible on the left.

FIG. 9 shows the NMR spectrum of PTMT− chemical structure indicating copolymerization of the three monomer units.

FIG. 10 shows SEM images of PTMT− coated on top of PS35 (left) and uncoated PS35 (right). The selective layer is visible on the left.

FIG. 11 shows the results of an oil-in-water 25 h fouling test of PTMS, PTMS−, and PTMS+. The initial flux of these membranes was set at 7.75 L/m²·h.

FIG. 12 shows the results of an oil-in-water 25 h fouling test of PTMM, and as a control Poly (2,2,2-trifluoroethyl methacrylate)-random-poly (methacrylic acid) (PTM−) with 41 mol % of TFEMA and 59 mol % of MAA. The initial flux of these membranes was set at 7.75 L/m²·h.

FIG. 13 shows the NMR spectrum of PTMS2 chemical structure indicating copolymerization of the three monomer units.

FIG. 14 shows SEM images of PTMS2 coated on top of PS35 (left) and uncoated PS35 (right).

FIG. 15 shows the NMR spectrum of PTMS3 chemical structure indicating copolymerization of the three monomer units.

FIG. 16 shows SEM images of PTMS3 coated on top of PS35 (left) and uncoated PS35 (right).

FIG. 17 shows the results of an oil-in-water 25 h fouling test of PTMS, PTMS2, and PTMS3. The initial flux of these membranes was set at 1.2 L/m²·h. NP030 is a commercial membrane of similar size cut-off and permeance.

FIG. 18 shows the results of 25 h protein fouling tests of PTMS2, and PTMS3; Lysozyme (left) and BSA (right). The initial flux of these membranes was set at 1 L/m²·h. NP030 is a commercial membrane of similar size cut-off and permeance.

FIG. 19 shows TEM bright-field images of r-PAC self-assembled morphologies with different −/+ charge ratio but similar hydrophobic monomer content, showing bicontinuous networks of ionic nanochannels (dark) surrounded by the hydrophobic phase (light). (Inset) FFT of the image with the arrow corresponding to the characteristic period of ionic domains. Panel (a) shows that PTMS exhibits a characteristic period of ˜3.1 nm, yielding a dry ionic channel size around 1.55 nm. Panel (b) shows that PTMS-exhibits a characteristic period of ˜4.5 nm, yielding a dry ionic channel size around 2.25 nm.

FIG. 20 is a graph showing modulated DSC analyses of PTMS, PTMS−, and PTMS+.

### DETAILED DESCRIPTION

This invention is based on the discovery of a novel family of terpolymers that combine hydrophobic, anionic, and cationic repeat units; and their use in fouling-resistant membrane filters with tunable selectivity. This invention, which combines hydrophobic, anionic, and cationic repeat units, represents the first ampholyte used in forming a selective membrane. The terpolymers of the invention, referred to herein as amphiphilic polyampholytes, comprise at least 1% each of a hydrophobic monomer, an anionic monomer, and a cationic monomer, copolymerized through a well-known polymerization reaction (e.g., free radical polymerization). The resultant polymers are insoluble in water.

These polymers can then be used to form coatings by common methods (e.g., doctor blading, bar coating, spray coating). These coatings can slow or prevent fouling. The fouling resistance is expected to be most effective when the anionic and cationic groups are as close to a 1:1 ratio by mole as possible, or when the surface is overall close to neutral in charge. Fouling is a major issue in many applications that involve aqueous solutions in contact with surfaces, including membrane filtration, preventing fouling on ship hulls and bridges, keeping pipes from clogging, biomaterials, etc.

When the coating is applied to a membrane with large pores, the resultant membranes exhibit not only high fouling resistance, but also selectivity that arises from the self-assembly of this polymer through interactions between different repeat units (e.g., Coulombic interactions between anionic and cationic groups). If the polymer contains close to a 1:1 ratio of anionic and cationic groups, the membranes exhibit roughly size-based selectivity with effective pore sizes in the range of <3 nm, along with excellent fouling resistance that is matched by few other systems. If there is an excess of one monomer, creating a net charge in the membrane layer, membranes will also have selectivity arising from electrostatic interactions/Donnan exclusion while maintaining good size-based selectivity. These membranes may be capable of exhibiting high rejections of various salts as well as charge-based selectivity (i.e., separating monovalent and divalent ions, separating dyes with different charges). These properties promise several applications in water and wastewater treatment, water purification, and bioseparations, as well as other aqueous separations.

The synthesis and coating processes of these membranes are highly scalable. This fact, combined with the several relevant applications listed, demonstrates their utility.

### Polymers

In certain aspects, the present invention provides a polymer, comprising:
(a) a plurality of hydrophobic repeat units;
(b) a plurality of a cationic repeat units; and
(c) a plurality of an anionic repeat units;

wherein the hydrophobic repeat units, cationic repeat units, and anionic repeat units are in random or statistical order.

In certain embodiments, the polymer is insoluble in water.

In certain embodiments, the molar ratio of cationic repeat units to anionic repeat units is about 1.1:1 to about 1:1.1, or about 1.05:1 to about 1:1.05, or about 1.01:1 to about 1:1.01. In certain preferred embodiments, the molar ratio of cationic repeat units to anionic repeat units is about 1:1. In certain embodiments, a ratio of cationic repeat units to anionic repeat units that is about 1:1 results in high fouling resistance.

In certain embodiments, the net charge of the polymer is from −10 to +10. In further embodiments, the net charge of the polymer is from −9 to +9, from −8 to +8, from −7 to +7, from −6 to +6, from −5 to +5, from −4 to +4, from −3 to +3, from −2 to +2, from −1 to +1, or is 0 (i.e., the polymer is neutral).

In certain embodiments, the molar ratio of cationic repeat units to anionic repeat units is greater than about 1:1, greater than about 1.25:1, greater than about 1.5:1, greater than about 1.75:1, or greater than about 2:1. In certain such embodiments, the net charge of the polymer is positive. In certain such embodiments, the net charge of the polymer is greater than +10.

In certain embodiments, the molar ratio of cationic repeat units to anionic repeat units is less than about 1:1, less than about 0.8:1; less than about 0.67:1, less than about 0.57:1, or less than about 0.5:1. In certain such embodiments, the net charge of the polymer is negative. In certain such embodiments, the net charge of the polymer is less than −10.

In certain embodiments, the molecular weight of the polymer is greater than about 20,000 g/mol, greater than about 40,000 g/mol, greater than about 60,000 g/mol, greater than about 80,000 g/mol, greater than about 100,000 g/mol, or greater than about 120,000 g/mol. In preferred embodiments, the molecular weight of the polymer is greater than about 100,000 g/mol.

The hydrophobic repeat unit limits the swelling of the polymer in water and imparts the polymer stability in aqueous environments. This hydrophobic repeat unit may be derived from a monomer whose homopolymer is not soluble in water, and has a glass transition temperature above use temperature (e.g., room temperature).

For use in the applications described herein (e.g., anti-fouling coatings and membrane selective layers), the polymers of the invention contain a sufficient amount of the hydrophobic repeat unit to be insoluble in water under use conditions.

In certain embodiments, the plurality of hydrophilic repeat units constitutes at least about 30 wt %, at least about 35 wt %, at least about 40 wt %, at least about 45 wt %, at least about 50 wt %, at least about 60 wt %, at least about 65 wt %, at least about 70 wt %, at least about 75 wt %, at least about 80 wt %, at least about 85 wt %, or at least about 90 wt % of the polymer. In preferred embodiments, the plurality of hydrophilic repeat units constitutes at least about 30 wt % or at least about 45% of the polymer to yield a polymer that is insoluble in water.

In certain embodiments, the plurality of hydrophilic repeat units constitutes about 30 wt % to about 99 wt %, about 30 wt % to about 90 wt %, about 40 wt % to about 90 wt %, about 40 wt % to about 80 wt %, about 40 wt % to about 75 wt %, about 40 wt % to about 70 wt %, about 45 wt % to about 75 wt %, about 45 wt % to about 70 wt %, about 50 wt % to about 80 wt %, about 50 wt % to about 75 wt %, or about 50 wt % to about 70 wt % of the polymer.

In certain embodiments, the plurality of hydrophilic repeat units constitutes about 40 wt % to about 90 wt % of the polymer. Certain such polymers may be used in anti-fouling coatings.

In certain embodiments, the plurality of hydrophilic repeat units constitutes about 40 wt % to about 75 wt %, preferably about 50 wt % to about 70 wt % of the polymer. Certain such polymers may be used in selective membrane layers.

In certain preferred embodiments of the polymers useful in anti-fouling coatings, these amphiphilic polyampholytes comprise ˜30-99 wt % (and preferably 40-90 wt %) of hydrophobic monomer and 0.5-40 wt % (and preferably 2.5-25 wt %) each of anionic and cationic monomers, with a molar ratio of anionic to cationic units as close to 1:1 as possible.

In certain embodiments, the plurality of cationic repeat units and the plurality of anionic repeat units each constitutes at least about 0.5 wt %, at least about 1 wt %, at least about 2.5 wt %, at least about 5 wt %, at least about 10 wt %, at least about 15 wt %, at least about 20 wt %, at least about 25 wt %, at least about 30 wt %, at least about 35 wt %, or at least about 40 wt % of the polymer.

In certain embodiments, the plurality of cationic repeat units and the plurality of anionic repeat units each constitutes about 0.5 wt % to about 30 wt %, about 0.5 wt % to about 25 wt %, about 1 wt % to about 30 wt %, about 1 wt % to about 25 wt %, about 2 wt % to about 30 wt %, about 2 wt % to about 25 wt %, about 2.5 wt % to about 30 wt %, about 2.5 wt % to about 25 wt %, about 5 wt % to about 30 wt %, or about 5 wt % to about 25 wt % of the polymer.

In certain embodiments, the plurality of hydrophobic repeat units comprises a mixture of different hydrophobic monomers.

For example, in some embodiments, each hydrophobic repeat unit is independently selected from the group consisting of 2,2-trifluoroethyl methacrylate (TFEMA), fluorinated acrylates, methacrylates, pentafluoropropyl methacrylate, heptafluorobutyl methacrylate, pentafluorophenyl methacrylate, styrene, methyl methacrylate, and acrylonitrile.

In further embodiments, each hydrophobic repeat unit is the same, and is selected from the group consisting of 2,2-trifluoroethyl methacrylate (TFEMA), fluorinated acrylates, methacrylates, pentafluoropropyl methacrylate, heptafluorobutyl methacrylate, pentafluorophenyl methacrylate, styrene, methyl methacrylate, and acrylonitrile. In certain preferred embodiments, the hydrophobic repeat unit is 2,2-trifluoroethyl methacrylate (TFEMA).

In some embodiments, the anionic repeat units carry a negative charge under use conditions. That is, in some embodiments, the anionic repeat units carry a negative charge in an aqueous solution at or above about pH 4.0, about pH 4.5, about pH 5.0, about pH 5.5, about pH 6.0, about pH 6.5, about pH 7.0, about pH 7.5, about pH 8.0, about pH 8.5, about pH 9.0, about pH 9.5, about pH 10.0, about pH 10.5, about pH 11.0, or about pH 11.5.

The anionic repeat unit may be selected from methacrylate, acrylate, methacrylamide, acrylamide, styrene, or vinyl derivatives containing a carboxylic acid, sulfonate, phosphate, or other ionizable or charged group that carries a negative charge under use conditions.

In certain embodiments, the plurality of anionic repeat units comprises a mixture of different anionic monomers.

For example, in some embodiments, each anionic repeat unit is independently selected from the group consisting of methacrylic acid (MAA), 2-sulfoethyl methacrylate (SEMA), L-tryptophan-methacrylamide (L-Try-MA), D-tryptophan-methacrylamide, L or D-tryptophan-acrylamide, L or D-alanine-methacrylamide, L or D-alanine-acrylamide, L or D-valine-methacrylamide, L or D-valine-acrylamide, L or D-isoleucine-methacrylamide, L or D-isoleucine-acrylamide, L or D-allo-isoleucine-methacrylamide, L or D-allo-isoleucine-acrylamide, L or D-methionine-methacrylamide, L or D-methionine-acrylamide, L or D-phenylalanine-methacrylamide, L or D-phenylalanine-acrylamide, L or D-tyrosine-methacrylamide, L or D-tyrosine-acrylamide, L or D-histadine-methacrylamide, L or D-histadine-acrylamide, L or D-glutamic acid-methacrylamide, L or D-glutamic acid-methacrylamide, L or D-aspartic acid-acrylamide, L or D-aspartic acid-methacrylamide, acrylic acid, 2-carboxyethyl acrylate, mono-2-(methacryloyloxy)ethyl succinate, mono-2-(methacryloyloxy)ethyl maleate, sodium 4-vinylbenzenesulfonate, 2-acrylamido-2-methylpropane sulfonic acid, 2-acrylamido-2-methyl-1-propanesulfonic acid sodium salt, 2-acrylamido-2-methyl-1-propanesulfonic acid, 3-sulfopropyl acrylate potassium salt, 3-sulfopropyl methacrylate potassium salt, 3-vinylbenzoic acid, 4-vinylbenzoic acid, 2-vinylbenzoic acid, 4-(2-propenyl)benzoic acid, 2-methyl-2-propene-1-sulfonic acid sodium salt, vinylsulfonic acid sodium salt, vinylphosphonic acid, and (4-ethenylphenyl)methylphosphonic acid.

In further embodiments, each anionic repeat unit is the same, and is selected from the group consisting of methacrylic acid (MAA), 2-sulfoethyl methacrylate (SEMA), L-tryptophan-methacrylamide (L-Try-MA), D-tryptophan-methacrylamide, L or D-tryptophan-acrylamide, L or D-alanine-methacrylamide, L or D-alanine-acrylamide, L or D-valine-methacrylamide, L or D-valine-acrylamide, L or D-isoleucine-methacrylamide, L or D-isoleucine-acrylamide, L or D-allo-isoleucine-methacrylamide, L or D-allo-isoleucine-acrylamide, L or D-methionine-methacrylamide, L or D-methionine-acrylamide, L or D-phenylalanine-methacrylamide, L or D-phenylalanine-acrylamide, L or D-tyrosine-methacrylamide, L or D-tyrosine-acrylamide, L or D-histadine-methacrylamide, L or D-histadine-acrylamide, L or D-glutamic acid-methacrylamide, L or D-glutamic acid-methacrylamide, L or D-aspartic acid-acrylamide, L or D-aspartic acid-methacrylamide, acrylic acid, 2-carboxyethyl acrylate, mono-2-(methacryloyloxy)ethyl succinate, mono-2-(methacryloyloxy)ethyl maleate, sodium 4-vinylbenzenesulfonate, 2-acrylamido-2-methylpropane sulfonic acid, 2-acrylamido-2-methyl-1-propanesulfonic acid sodium salt, 2-acrylamido-2-methyl-1-propanesulfonic acid, 3-sulfopropyl acrylate potassium salt, 3-sulfopropyl methacrylate potassium salt, 3-vinylbenzoic acid, 4-vinylbenzoic acid, 2-vinylbenzoic acid, 4-(2-propenyl)benzoic acid, 2-methyl-2-propene-1-sulfonic acid sodium salt, vinylsulfonic acid sodium salt, vinylphosphonic acid, and (4-ethenylphenyl)methylphosphonic acid.

In certain such embodiments, the anionic repeat unit is selected from the group consisting of methacrylic acid (MAA), 2-sulfoethyl methacrylate (SEMA), and L-tryptophan-methacrylamide (L-Try-MA). In preferred embodiments, the anionic repeat unit is methacrylic acid (MAA). In other preferred embodiments, the anionic repeat unit is 2-sulfoethyl methacrylate (SEMA).

In some embodiments, the cationic repeat units carry a positive charge under use conditions. That is, in some embodiments, the cationic repeat units carry a positive charge in an aqueous solution at or below about pH 1.0, about pH 1.5, about pH 2.0, about pH 2.5, about pH 3.0, about pH 3.5, about pH 4.0, about pH 4.5, about pH 5.0, about pH 5.5, about pH 6.0, about pH 6.5, about pH 7.0, about pH 7.5, about pH 8.0, about pH 8.5, about pH 9.0, or about pH 9.5.

The cationic repeat unit may be selected from methacrylate, acrylate, methacrylamide, acrylamide, styrene, or vinyl derivatives containing amine, ammonium, pyridinium, imidazolium, phosphonium, pyrrolidinium, or other ionizable or charged groups that carry a positive charge under use conditions. In preferred embodiments, the cationic repeat unit has a strong cationic charge such as a quaternary ammonium group.

In certain embodiments, the plurality of cationic repeat units comprises a mixture of different cationic monomers.

For example, in some embodiments, each cationic repeat unit is independently selected from the group consisting of [2-(methacryloyloxy)ethyl]trimethylammonium chloride (MAETA), [3-(methacryloylamino)propyl]trimethylammonium chloride, [2-(acryloyloxy)ethyl]trimethylammonium chloride, (3-acrylamidopropyl)trimethylammonium chloride, 4-vinylbenzyl(triphenyl)phosphonium chloride, (vinylbenzyl)trimethylammonium chloridemethacrylate, 3-vinylaniline, 4-vinylaniline, 2-isopropenylaniline, N-(3-aminopropyl) methacrylamide hydrochloride, N-vinylimidazolium salts, 1-allyl-3-vinylimidazolium salts, 1-allyl-2methyl-5-vinyl-pyridinium salt, 1-allyl-5-vinyl-pyridinium salt, and 1-methyl-1-(1-vinylcyclohexyl) pyrrolidinium iodide.

In further embodiments, each cationic repeat unit is the same, and is selected from the group consisting of [2-(methacryloyloxy)ethyl]trimethylammonium chloride (MAETA), [3-(methacryloylamino)propyl]trimethylammonium chloride, [2-(acryloyloxy)ethyl]trimethylammonium chloride, (3-acrylamidopropyl)trimethylammonium chloride, 4-vinylbenzyl(triphenyl)phosphonium chloride, (vinylbenzyl)trimethylammonium chloridemethacrylate, 3-vinylaniline, 4-vinylaniline, 2-isopropenylaniline, N-(3-aminopropyl) methacrylamide hydrochloride, N-vinylimidazolium salts, 1-allyl-3-vinylimidazolium salts, 1-allyl-2methyl-5-vinyl-pyridinium salt, 1-allyl-5-vinyl-pyridinium salt, and 1-methyl-1-(1-vinylcyclohexyl) pyrrolidinium iodide.

In certain embodiments, the cationic repeat unit is 2-(methacryloyloxy)ethyl]trimethylammonium chloride (MAETA).

In certain preferred embodiments, the cationic repeat unit comprises a quaternary ammonium group.

In certain embodiments, the polymer consists essentially of or consists of:
(a) a plurality of hydrophobic repeat units;
(b) a plurality of a cationic repeat units; and
(c) a plurality of an anionic repeat units;
wherein the hydrophobic repeat units, cationic repeat units, and anionic repeat units are in random or statistical order.

The polymers of the invention may be made in accordance with the examples provided herein. The polymers may be synthesized from vinyl monomers (e.g., acrylates, methacrylates, acrylamides, styrene derivatives, acrylonitrile) using well-known polymerization methods (e.g., free radical polymerization).

The polymers described herein have not been previously prepared or studied. While combinations of anionic and cationic monomers have been used as anti-fouling coatings, the incorporation of hydrophobic groups into such known materials would have been expected to decrease fouling resistance; yet, the resultant materials exhibit fouling resistance comparable to or even exceeding all-hydrophilic coatings.

The self-assembly of polyampholytes, let alone amphiphilic polyampholytes with additional functional groups, is not well-studied at all. Interactions between repeat units of amphiphilic polyampholytes are unlikely to be the same as those observed in zwitterionic amphiphilic copolymers (ZACs).

Additionally, the present invention is advantageous because ionic or ionizable monomers are more widely available, cheaper, and typically easier to solubilize compared with zwitterionic monomers. Furthermore, the abundance of monomer choices allows for multiple combinations of anionic and cationic units with potential different membrane performances, including amino acid-based monomers for chiral and other bioseparations.

The term “polyampholyte” as used herein is a polymer that contains both anionically and cationically charged repeat units. They are typically very hydrophilic and often water-soluble in the absence of any non-ionic groups. As such, the polymers described in this invention are referred to as “amphiphilic polyampholytes” due to the presence of both hydrophobic and ampholytic segments on the same backbone.

The term “charged” as used herein is used to describe a species that carries an overall deficiency or excess of electrons relative to the total valence electron count of the species.

The term “anionic” as used herein refers to a species with an overall excess of electrons relative to the total valence electron count of the species.

The term “cationic” as used herein refers to a species with an overall deficiency of electrons relative to the total valence electron count of the species.

The term “net charge” as used herein refers to the total overall charge on a polymer containing cationic and anionic moieties. The net charge is calculated as: (number of positive charges)−(number of negative charges).

The term “statistical order” refers to a polymer in which the sequence of the constituent repeat units follows a statistical rule. Statistical order is dictated by the reaction kinetics of the chemically distinct monomer reactants (or repeat unit reactants). In some embodiments, statistical order encompasses random order.

A polymer with “random order” (alternatively referred to as a “random copolymer”) is a polymer in which the probability of finding a given type of repeat unit at a particular point in the chain is equal to the mole fraction of that repeat unit in the polymer.

### Membranes

The novel polymeric materials of the invention form highly fouling-resistant surfaces with controlled surface charge. Thus, the polymeric materials are useful in the preparation of membranes useful for anti-fouling applications. The membranes may be prepared by coating these polymeric materials onto porous supports, which offers fouling resistance as well as controlled selectivity corresponding to effective pore sizes <4 nm, and preferably between 0.5-2 nm, coupled with tunable charge-based selectivity through the incorporation of excess charge into the polymer. This invention has uses in nanofiltration, ultrafiltration, and reverse osmosis applications.

Anti-fouling coatings can be applied on various surfaces (e.g., pipes, ship hulls, outside of surfaces immersed in water, biomedical materials) using well-known methods (e.g., spray coating, roll coating, painting). They are expected to prevent the adsorption of bioorganic macromolecules (e.g., proteins, alginate, natural organic matter) as well as oil onto the surface in water.

Another application of these amphiphilic polyampholytes is as selective, fouling-resistant membrane selective layers. For use as membrane selective layers, the polymer needs to be insoluble in water, but permeable to it. As such, preferred compositions contain lower hydrophobic repeat unit content (e.g., 40-75 wt %; preferably 50-70 wt %), with the rest being a combination of anionic and cationic repeat units. The ratio of these charged groups will affect solute selectivity.

In some embodiments, the invention provides a thin film composite membrane, comprising a porous substrate, and a selective layer comprising a polymer of the invention, wherein an average effective pore size of the porous substrate is larger than an average effective pore size of the selective layer; and the selective layer is disposed on a surface of the porous substrate.

In some embodiments, the selective layer has a thickness of about 10 nm to about 10 μm, or about 10 nm to about 3 μm, or about 10 nm to about 1 μm.

In some embodiments, the selective layer has the average effective pore size of less than about 4 nm. In further embodiments, the selective layer has the average effective pore size of about 0.1 nm to about 2.0 nm, about 0.5 nm to about 2.0 nm, about 0.1 nm to about 1.2 nm, about 0.5 nm to about 1.2 nm, or about 0.7 nm to about 1.2 nm.

In some embodiments, the net charge of the polymer is positive, and the thin film composite membrane rejects positively charged solutes and salts. Alternatively, in some embodiments, the net charge of the polymer is negative, and the thin film composite membrane rejects negatively charged solutes and salts.

In some embodiments, the net charge of the polymer is from −10 to +10; and the selective layer exhibits size-based selectivity between uncharged organic molecules. In certain such embodiments, the net charge of the polymer is from −9 to +9, from −8 to +8, from −7 to +7, from −6 to +6, from −5 to +5, from −4 to +4, from −3 to +3, from −2 to +2, from −1 to +1, or is 0 (i.e., the polymer is neutral).

In certain embodiments, the selective layer exhibits rejection of greater than or equal to about 95% or greater than or equal to about 99% for neutral molecule with hydrated diameter of about or greater than 1.5 nm.

In certain embodiments, the selective layer exhibits antifouling properties.

In certain embodiments, the selective layer exhibits resistance to fouling by an oil emulsion.

In certain embodiments, the selective layer exhibits resistance to fouling by adsorption of bioorganic macromolecules (e.g., proteins, alginate, organic matter).

The membranes are prepared by coating methods well-established in the field. For example, the polymer is dissolved in an appropriate solvent. The solution is coated onto a porous support (e.g., a membrane with large pores in the microfiltration or ultrafiltration range) using established methods (e.g., doctor blading, bar coating, spray coating). The solvent is evaporated. It certain preferred embodiments, the coated membrane is immersed in water or another non-solvent to accelerate precipitation. The product is a thin film composite (TFC) membrane, comprising at least two layers: A porous support with large pores, providing mechanical integrity, and a thin (preferably <10 μm, more preferably <3 μm, even more preferably <1 μm) layer of the polymer, which serves as the “selective layer” of the membrane.

The resultant membranes exhibit roughly size-based separation of neutral organic molecules. If the anionic and cationic groups are at approximately a 1:1 molar ratio, the membrane will be mostly size-selective. These membranes are also more fouling-resistant.

Different molar charge ratios will lead to the rejection of solutes based on their charge, leading to higher rejection of charged solutes than neutral solutes of the same size, as well as increased salt rejection. The charge ratio can also affect effective pore size, likely by altering how the polymer self-assembles due to the Coulombic interactions during membrane formation. The data below indicate polymers with an excess of one charge have higher effective pore sizes.

All amphiphilic polyampholyte TFC membranes tested to date exhibit excellent fouling resistance comparable with the best in the field, such as those manufactured from zwitterionic amphiphilic copolymers (ZACs).³⁴⁻³⁹ All membranes tested (see examples below) have shown no measurable irreversible fouling.

The pore size and net charge of these membranes may be tuned by utilizing different monomers, different monomer ratios, and monomers with cross linkable groups (e.g., 1-allyl-3-vinylimidazolium salts; 1-allyl-2-methyl-5-vinyl-pyridinium salt; 1-allyl-5-vinyl-pyridinium salt).

In further embodiments, the invention provides a material with an anti-fouling coating, comprising a solid substrate, and a layer comprising a polymer of the invention, wherein the layer is disposed on a surface of the solid substrate.

In certain embodiments, the layer has a thickness of about 10 nm to about 10 μm, or about 10 nm to about 3 μm, or about 10 nm to about 1 μm.

### Methods of Use

The membranes described herein are useful in various applications, particularly in aqueous separation applications, including water and wastewater treatment, bioseparations, water reuse, and purification and concentration of pharmaceutical products. The wastewater treatment applications encompass water softening applications, and treatment of complex, high fouling water streams, water streams containing some solvent and/or at high temperatures that would damage un-cross-linked membrane materials, and frac or produced water from oil and gas extraction, which is hot with high salinity.

In some embodiments, the membranes described herein may be used in water and wastewater treatment applications. In certain such embodiments, a contaminated solution may be filtered through a thin film composition (TFC) membrane, whereby one or more contaminants are retained by the TFC membrane and removed from the solution. In certain such embodiments, the fouling resistance of the membrane enables the solution to retain a stable flux through the membrane. The undesired contaminants may be, for example, organic solutes. In further embodiments, the undesired contaminants may be a fragmented antibody, aggregated antibodies, a host cell protein, a polynucleotide, an endotoxin, or a virus.

In a related use, the membranes described herein may be used in water reuse applications, in which a contaminated water stream is filtered through the TFC membrane, whereby one or more contaminants are retained by the TFC membrane, and the filtrate is reused, optionally within the same facility and optionally after further treatment.

Accordingly, in certain embodiments, the invention provides a method of removing an organic solute from water, comprising contacting at a first flow rate a thin film composite membrane described herein with an aqueous solution comprising an organic solute, whereby the organic solute is retained by the thin film composite membrane.

In certain embodiments, substantially all of the organic solute is retained by the thin film composite membrane.

In further embodiments, the invention provides a method of decontaminating water, comprising contacting at a first flow rate the thin film composite membrane described herein with an mixture comprising water and one or more contaminants, whereby one or more contaminants are retained by the thin film composite membrane.

In certain embodiments, substantially all of the contaminants are retained by the thin film composite membrane.

In certain embodiments, the flow path of the aqueous solution or the mixture comprising water is substantially through macropores of the thin film composite membrane.

In certain embodiments, the invention relates to any one of the aforementioned methods, further comprising the steps of:
cleaning the thin film composite membrane; and
repeating the above-mentioned steps.

In further embodiments, the membranes described herein may be used in bioseparations applications. In such embodiments, the thin film composite membrane preferably has a sufficiently low pore size to retain peptide drugs and other lower molar mass biopharmaceuticals, while allowing the removal of small molecule solutes and/or salts.

Accordingly, in certain embodiments, the invention provides a method of purifying a peptide or protein, comprising contacting at a first flow rate a thin film composite membrane of the invention with a mixture comprising one or more peptides or proteins, whereby a portion of the mixture is retained by the thin film composite membrane.

In certain embodiments, the portion of the mixture that is retained by the TFC membrane comprises one or more peptides or proteins. In certain such embodiments, one or more contaminants presents in the initial mixture comprising one or more peptides or proteins is not retained by the TFC membrane, but rather flows through the membrane. This process therefore separates the desired peptides or proteins from the undesired contaminants.

In certain embodiments, substantially all of the one or more peptides or proteins are retained by the thin film composite membrane.

The method may further comprise a step of releasing the retained peptide or protein from the TFC membrane. In such embodiments, the method further comprises contacting the protein or peptide retained by the TFC membrane with a second fluid at a second flow rate, thereby releasing a portion of the retained peptide or protein from the TFC membrane.

The second fluid may be a buffer. The second fluid may comprise a salt.

The membranes described herein are also useful in size-selection separation applications. The pore size and net charge of the membranes may be tuned according to the desired utility of the TFC membranes, e.g., by selecting different monomers, different monomer ratios, or by cross-linking different functional groups onto the constituent monomers.

Accordingly, in some embodiments, the invention provides a method of size-selective separation, comprising contacting at a first flow rate the thin film composite membrane described herein with a mixture comprising one or more particles of differing sizes, whereby one or more particles of a particular size are retained by the thin film composite membrane.

### EXAMPLES

The disclosure will be further illustrated with reference to the following specific examples. These examples are given by way of illustration and are not meant to limit the disclosure or any claims.

### Example 1. Synthesis of Poly (2,2,2-trifluoroethyl methacrylate)-random-poly (2-(methacryloyloxy)ethyl]trimethylammonium chloride)-random-poly (2-sulfoethyl methacrylate) (PTMS)

In this example, a random terpolymer was prepared with the three monomeric units being: 2-sulfoethyl methacrylate (SEMA, Polysciences), [2-(methacryloyloxy)ethyl]trimethylammonium chloride (MAETA, Sigma Aldrich), and 2,2,2-trifluoroethyl methacrylate (TFEMA, ACROS Organics). The first two monomers were passed through a column of neutral activated alumina (Sigma Aldrich) to remove the inhibitor, while the last monomer was passed through a column of basic activated alumina (Sigma Aldrich). SEMA (2.42 g, 12.4 mmol), MAETA (2.58 g, 12.4 mmol), and 2,2,2-trifluoroethyl methacrylate (5.00 g, 29.7 mmol) were dissolved in this order in dimethyl sulfoxide (DMSO, 40 mL). Azobisisobutyronitrile (AIBN, 0.01 g, Aldrich) was added to the flask. The flask was sealed with a rubber septum. Nitrogen was bubbled through the reaction mixture for 30 minutes to purge any dissolved oxygen. The flask was then kept at 60° C., while stirring at 300 rpm for 17 hours. After the reaction, 1 g of 4-methoxyphenol (MEHQ) was added to terminate the synthesis. The reaction mixture, which was observed to be viscous, was then precipitated in acetone, purified by stirring two fresh portions of 1:5 methanol to acetone volume ratio for at least 5 hours, and two fresh portions of acetone for at least 5 hours. Finally, the terpolymer was dried in the vacuum oven for 72 h at 60° C. The composition of the terpolymer was calculated from the 1H-NMR spectrum (FIG. 1), using the ratio of protons from the three different monomer units. The calculated composition was: 22 mol % SEMA, 22 mol % MAETA, and 56 mol % TFEMA. The final conversion of this reaction was 45%.

### Example 2. Formation of Thin Film Composite Membranes from PIMS

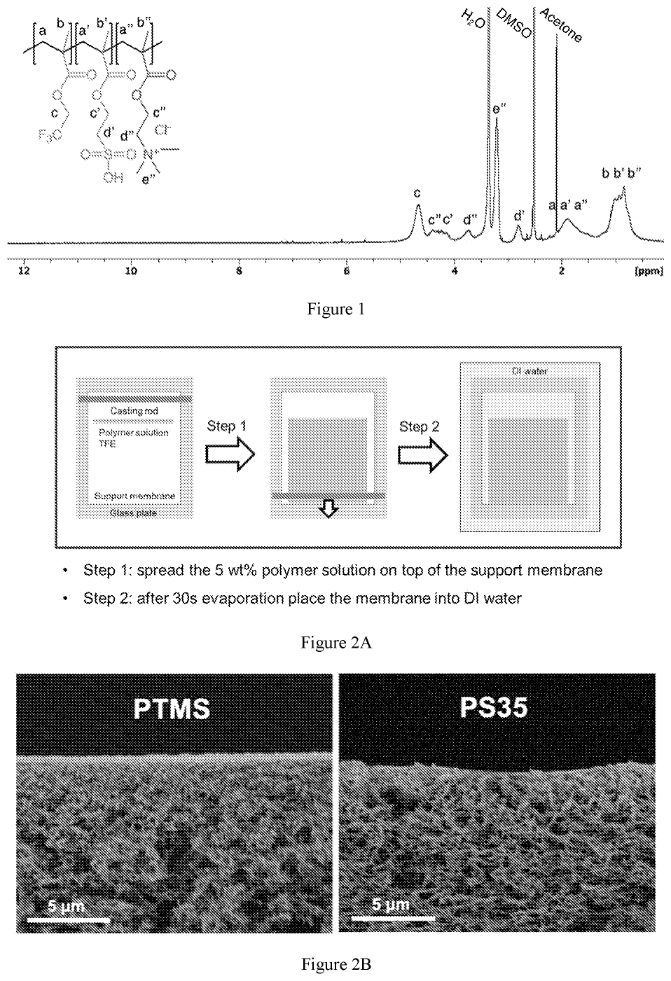

In this example, a membrane was prepared using the polymer described in example 1. A schematic is shown in FIG. 2a. The terpolymer (0.5 g) was dissolved in trifluoroethanol (TFE, 9.5 g) at approximately 25° C. The terpolymer solution was passed through a 0.45 micrometer syringe filter (Whatman) and degassed in a vacuum oven for at least 1 hours. The membranes were prepared by coating a thin layer of terpolymer solution on a commercial ultrafiltration (UF) membrane using a film applicator rod. PS35 ultrafiltration membrane, purchased from Solecta (Oceanside, Calif.), was used as the base membrane. After coating, the membrane was immersed in a water bath.

Film thickness and morphology was determined by the examination of freeze-fractured cross-sections of the membranes using a scanning electron microscope (SEM, Phenom G2 Pure Tabletop SEM). In FIG. 2b, SEM images of the uncoated base membrane (right) and the coated membrane (left) are given at the same magnification. The coating layer can be observed, it is dense (i.e. without macroscopic pores) with a thickness of about 0.7 micrometers.

### Example 3. Synthesis of Poly (2,2,2-trifluoroethyl methacrylate)-random-poly (2-(methacryloyloxy)ethyl]trimethylammonium chloride)-random-poly (2-sulfoethyl methacrylate) (PTMS−)

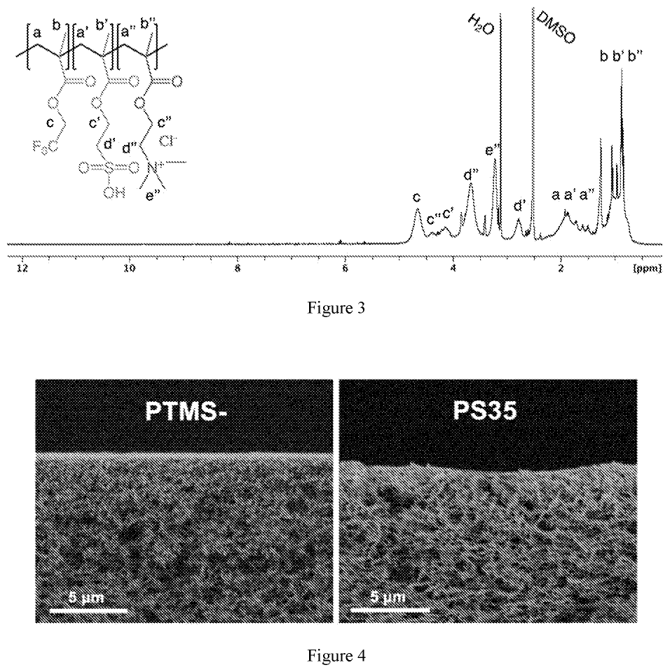

In this example, a random terpolymer was prepared with the three monomeric units being: 2-sulfoethyl methacrylate (SEMA, Polysciences), [2-(methacryloyloxy)ethyl]trimethylammonium chloride (MAETA, Sigma Aldrich), and 2,2,2-trifluoroethyl methacrylate (TFEMA, ACROS Organics). The first two monomers were passed through a column of neutral activated alumina (Sigma Aldrich) to remove the inhibitor, while the last monomer was passed through a column of basic activated alumina (Sigma Aldrich). SEMA (3.39 g, 17.5 mmol), MAETA (1.55 g, 7.5 mmol), and 2,2,2-trifluoroethyl methacrylate (4.94 g, 29.4 mmol) were dissolved in this order in dimethyl sulfoxide (DMSO, 40 mL). Azobisisobutyronitrile (AIBN, 0.01 g, Aldrich) was added to the flask. The flask was sealed with a rubber septum. Nitrogen was bubbled through the reaction mixture for 30 minutes to purge any dissolved oxygen. The flask was then kept at 60° C., while stirring at 300 rpm for 17 hours. After the reaction, 1 g of 4-methoxyphenol (MEHQ) was added to terminate the synthesis. The reaction mixture, which was observed to be viscous, was then precipitated in acetone, purified by stirring two fresh portions of 1:3 ethanol to hexane volume ratio for at least 8 hours. Finally, the terpolymer was dried in the vacuum oven for 72 h at 60° C. The composition of the terpolymer was calculated from the 1H-NMR spectrum (FIG. 3), using the ratio of protons from the three different monomer units. The calculated composition was: 28 mol % SEMA, 16 mol % MAETA, and 56 mol % TFEMA. The final conversion of this reaction was 58%.

### Example 4. Formation of Thin Film Composite Membranes from PTMS−

In this example, a membrane was prepared using the polymer described in example 3. The terpolymer (0.5 g) was dissolved in trifluoroethanol (TFE, 9.5 g) at approximately 25° C. The terpolymer solution was passed through a 0.45 micrometer syringe filter (Whatman) and degassed in a vacuum oven for at least 1 hours. The membranes were prepared by coating a thin layer of terpolymer solution on a commercial ultrafiltration (UF) membrane using a film applicator rod. PS35 ultrafiltration membrane, purchased from Solecta (Oceanside, Calif.), was used as the base membrane. After coating, the membrane was immersed in a water bath.

Film thickness and morphology was determined by the examination of freeze-fractured cross-sections of the membranes using a scanning electron microscope (SEM, Phenom G2 Pure Tabletop SEM). In FIG. 4, SEM images of the uncoated base membrane (right) and the coated membrane (left) are given at the same magnification. The coating layer can be observed, it is dense (i.e. without macroscopic pores) with a thickness of about 0.7 micrometers.

### Example 5. Synthesis of Poly (2,2,2-trifluoroethyl methacrylate)-random-poly (2-(methacryloyloxy)ethyl]trimethylammonium chloride)-random-poly (2-sulfoethyl methacrylate) (PTMS+)

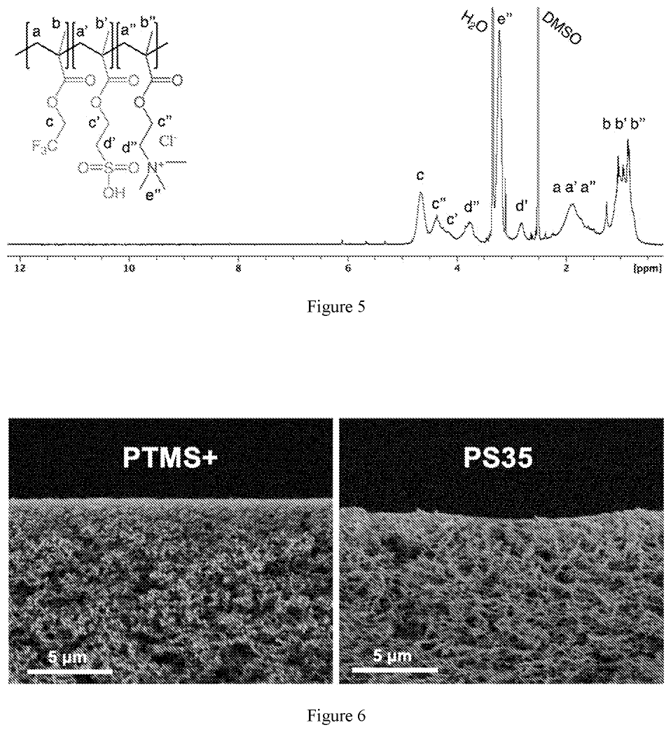

In this example, a random terpolymer was prepared with the three monomeric units being: 2-sulfoethyl methacrylate (SEMA, Polysciences), [2-(methacryloyloxy)ethyl]trimethylammonium chloride (MAETA, Sigma Aldrich), and 2,2,2-trifluoroethyl methacrylate (TFEMA, ACROS Organics). The first two monomers were passed through a column of neutral activated alumina (Sigma Aldrich) to remove the inhibitor, while the last monomer was passed through a column of basic activated alumina (Sigma Aldrich). SEMA (1.45 g, 7.5 mmol), MAETA (3.61 g, 17.4 mmol), and 2,2,2-trifluoroethyl methacrylate (5.06 g, 30.1 mmol) were dissolved in this order in dimethyl sulfoxide (DMSO, 40 ml). Azobisisobutyronitrile (AIBN, 0.01 g, Aldrich) was added to the flask. The flask was sealed with a rubber septum. Nitrogen was bubbled through the reaction mixture for 30 minutes to purge any dissolved oxygen. The flask was then kept at 60° C., while stirring at 300 rpm for 17 hours. After the reaction, 1 g of 4-methoxyphenol (MEHQ) was added to terminate the synthesis. The reaction mixture, which was observed to be viscous, was then precipitated in a 1:1 hexane to acetone volume ratio, purified by stirring two fresh portions of 1:3 ethanol to hexane volume ratio for at least 8 hours. Finally, the terpolymer was dried in the vacuum oven for 72 h at 60° C. The composition of the terpolymer was calculated from the ¹H-NMR spectrum (FIG. 5), using the ratio of protons from the three different monomer units. The calculated composition was: 15 mol % SEMA, 29 mol % MAETA, and 56 mol % TFEMA. The final conversion of this reaction was 66%.

### Example 6. Formation of Thin Film Composite Membranes from PTMS+

In this example, a membrane was prepared using the polymer described in example 5. The terpolymer (0.5 g) was dissolved in trifluoroethanol (TFE, 9.5 g) at approximately 25° C. The terpolymer solution was passed through a 0.45 micrometer syringe filter (Whatman) and degassed in a vacuum oven for at least 1 hours. The membranes were prepared by coating a thin layer of terpolymer solution on a commercial ultrafiltration (UF) membrane using a film applicator rod. PS35 ultrafiltration membrane, purchased from Solecta (Oceanside, Calif.), was used as the base membrane. After coating, the membrane was immersed in a water bath.

Film thickness and morphology was determined by the examination of freeze-fractured cross-sections of the membranes using a scanning electron microscope (SEM, Phenom G2 Pure Tabletop SEM). In FIG. 6, SEM images of the uncoated base membrane (right) and the coated membrane (left) are given at the same magnification. The coating layer can be observed, it is dense (i.e. without macroscopic pores) with a thickness of about 0.6 micrometers.

### Example 7. Synthesis of Poly (2,2,2-trifluoroethyl methacrylate)-random-poly (2-(methacryloyloxy)ethyl]trimethylammonium chloride)-random-poly (methacrylic acid) (PTMM)

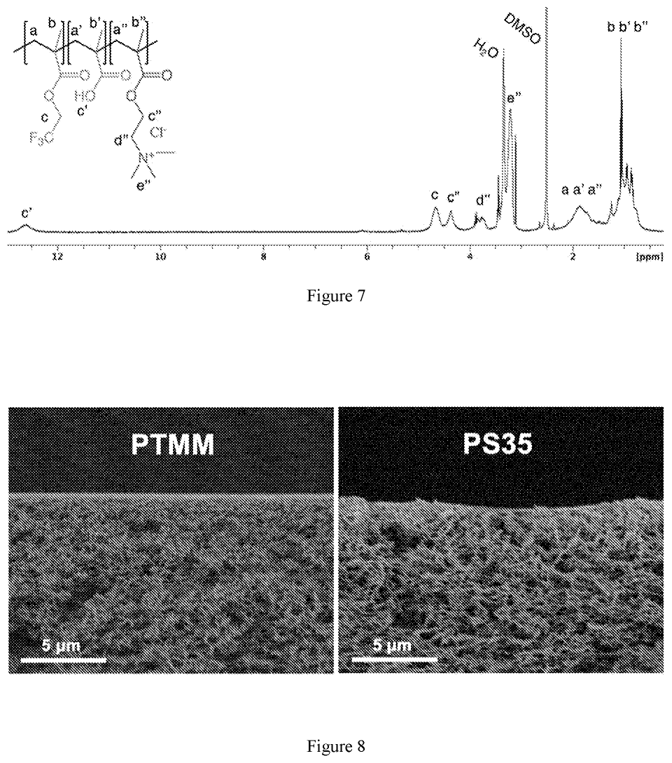

In this example, a random terpolymer was prepared with the three monomeric units being: methacrylic acid (MAA, Sigma Aldrich), [2-(methacryloyloxy)ethyl]trimethylammonium chloride (MAETA, Sigma Aldrich), and 2,2,2-trifluoroethyl methacrylate (TFEMA, ACROS Organics). The first two monomers were passed through a column of neutral activated alumina (Sigma Aldrich) to remove the inhibitor, while the last monomer was passed through a column of basic activated alumina (VWR). MAA (1.46 g, 17.0 mmol), MAETA (3.54 g, 17.0 mmol), and 2,2,2-trifluoroethyl methacrylate (5.00 g, 29.7 mmol) were dissolved in this order in dimethyl sulfoxide (DMSO, 40 mL). Azobisisobutyronitrile (AIBN, 0.01 g, Aldrich) was added to the flask. The flask was sealed with a rubber septum. Nitrogen was bubbled through the reaction mixture for 30 minutes to purge any dissolved oxygen. The flask was then kept at 60° C., while stirring at 300 rpm for 17 hours. After the reaction, 1 g of 4-methoxyphenol (MEHQ) was added to terminate the synthesis. The reaction mixture, which was observed to be viscous, was then precipitated in acetone, purified by stirring two fresh portions of 1:3 ethanol to hexane volume ratio for at least 5 hours, and two fresh portions of acetone for at least 5 hours. Finally, the terpolymer was dried in the vacuum oven for 72 h at 60° C. The composition of the terpolymer was calculated from the ¹H-NMR spectrum (FIG. 7), using the ratio of protons from the three different monomer units. The calculated composition was: 25 mol % MAA, 25 mol % MAETA, and 50 mol % TFEMA. The final conversion of this reaction was 57%.

### Example 8. Formation of Thin Film Composite Membranes from PTMM

In this example, a membrane was prepared using the polymer described in example 7. The terpolymer (0.5 g) was dissolved in methanol (MeOH, 9.5 g) at approximately 25° C. The terpolymer solution was passed through a 0.45 micrometer syringe filter (Whatman) and degassed in a vacuum oven for at least 1 hours. The membranes were prepared by coating a thin layer of terpolymer solution on a commercial ultrafiltration (UF) membrane using a film applicator rod. PS35 ultrafiltration membrane, purchased from Solecta (Oceanside, Calif.), was used as the base membrane. After coating, the membrane was immersed in a water bath.

Film thickness and morphology was determined by the examination of freeze-fractured cross-sections of the membranes using a scanning electron microscope (SEM, Phenom G2 Pure Tabletop SEM). In FIG. 8, SEM images of the uncoated base membrane (right) and the coated membrane (left) are given at the same magnification. The coating layer can be observed, it is dense (i.e. without macroscopic pores) with a thickness of about 0.4 micrometers.

### Example 9. Synthesis of Poly (2,2,2-trifluoroethyl methacrylate)-random-poly (2-(methacryloyloxy)ethyl]trimethylammonium chloride)-random-poly (L-tryptophan-methacrylamide) (PTMT−)

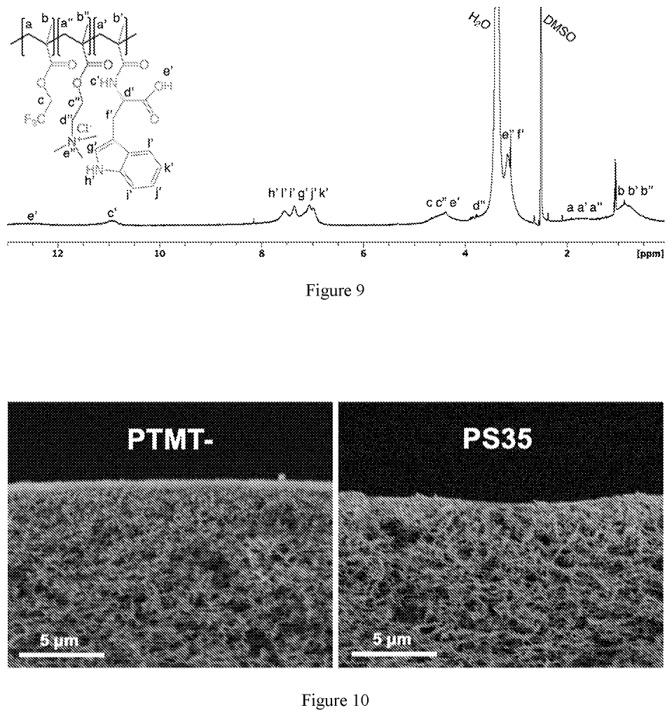

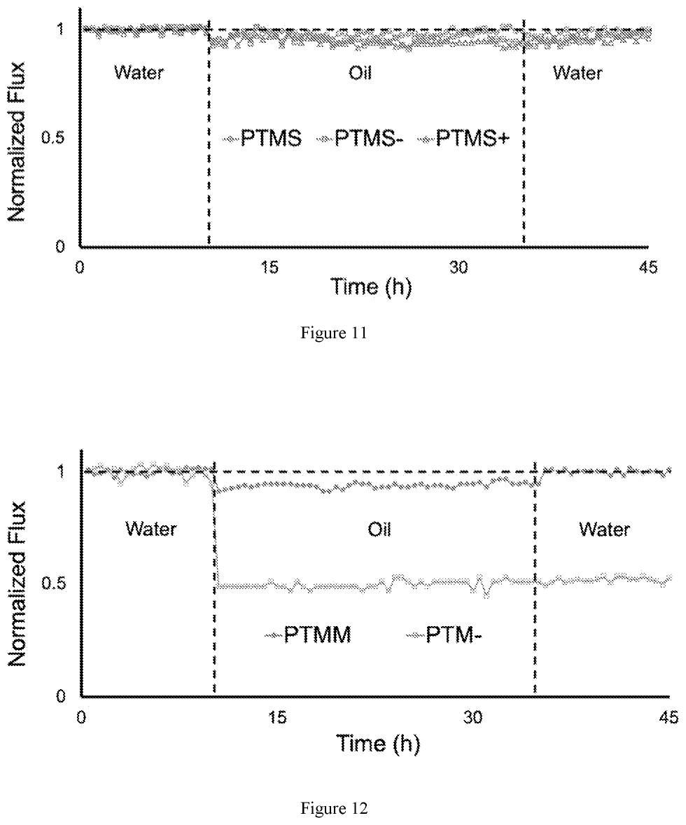

In this example, a random terpolymer was prepared with the three monomeric units being: L-tryptophan-methacrylamide (L-Try-MA, synthesized), [2-(methacryloyloxy)ethyl]trimethylammonium chloride (MAETA, Sigma Aldrich), and 2,2,2-trifluoroethyl methacrylate (TFEMA, ACROS Organics). MAETA was passed through a column of neutral activated alumina (Sigma Aldrich) to remove the inhibitor, while the TFEMA was passed through a column of basic activated alumina (Sigma Aldrich). L-Try-MA (1.18 g, 4.2 mmol), MAETA (0.37 g, 1.8 mmol), and 2,2,2-trifluoroethyl methacrylate (1.55 g, 9.2 mmol) were dissolved in this order in dimethyl sulfoxide (DMSO, 11.3 mL). Azobisisobutyronitrile (AIBN, 0.003 g, Aldrich) was added to the flask. The flask was sealed with a rubber septum. Nitrogen was bubbled through the reaction mixture for 30 minutes to purge any dissolved oxygen. The flask was then kept at 60° C., while stirring at 300 rpm for 17 hours. After the reaction, 0.3 g of 4-methoxyphenol (MEHQ) was added to terminate the synthesis. The reaction mixture, which was observed to be viscous, was then precipitated in acetone, purified by stirring four fresh portions of 1:2 ethanol to hexane volume ratio for at least 5 hours. Finally, the terpolymer was dried in the vacuum oven for 72 h at 60° C. The composition of the terpolymer was calculated from the ¹H-NMR spectrum (FIG. 9), using the ratio of protons from the three different monomer units. The calculated composition was: 34 mol % L-Try-MA, 15 mol % MAETA, and 51 mol % TFEMA. The final conversion of this reaction was 30%.

### Example 10. Formation of Thin Film Composite Membranes from PTMT−

In this example, a membrane was prepared using the polymer described in example 9. The terpolymer (0.2 g) was dissolved in DMSO (3.8 g) at approximately 25° C. The terpolymer solution was passed through a 0.45 micrometer syringe filter (Whatman) and degassed in a vacuum oven for at least 1 hours. The membranes were prepared by coating a thin layer of terpolymer solution on a commercial ultrafiltration (UF) membrane using a film applicator rod. PS35 ultrafiltration membrane, purchased from Solecta (Oceanside, Calif.), was used as the base membrane. After coating, the membrane was immersed in a water bath.

Film thickness and morphology was determined by the examination of freeze-fractured cross-sections of the membranes using a scanning electron microscope (SEM, Phenom G2 Pure Tabletop SEM). In FIG. 10, SEM images of the uncoated base membrane (right) and the coated membrane (left) are given at the same magnification. The coating layer can be observed, it is dense (i.e. without macroscopic pores) with a thickness of about 0.6 micrometers.

### Example 11. Fouling Resistance of Polyampholyte Terpolymer Membranes

In this example, the membranes prepared as described in examples 2, 4, 6, and 8 were used in experiments to determine their fouling resistance properties. The retention experiments were performed on an Amicon 8010 stirred, dead-end filtration cell (Millipore) with a cell volume of 10 mL and an effective filtration area of 4.1 cm². The cell was stirred at 500 rpm. The feed solution consisted of a 1500 ppm oil-in-water emulsion (9:1 ratio of soybean oil:DC 193 surfactant obtained from Dow-Corning), prepared by blending the oil, water, and surfactant using a blender in high rpm for ˜3 min.

### Example 12. Synthesis of Poly (2,2,2-trifluoroethyl methacrylate)-random-poly (2-(methacryloyloxy)ethyl]trimethylammonium chloride)-random-poly (2-sulfoethyl methacrylate) (PTMS2)

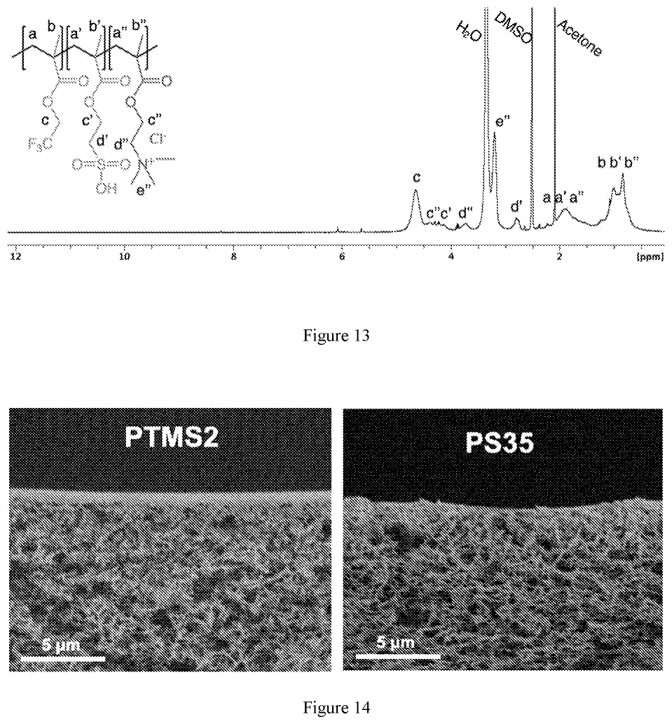

In this example, a random terpolymer with the three monomeric units being: 2-sulfoethyl methacrylate (SEMA, Polysciences), [2-(methacryloyloxy)ethyl]trimethylammonium chloride (MAETA, Sigma Aldrich), and 2,2,2-trifluoroethyl methacrylate (TFEMA, ACROS Organics). The first two monomers were passed through a column of neutral activated alumina (Sigma Aldrich) to remove the inhibitor, while the last monomer was passed through a column of basic activated alumina (Sigma Aldrich). SEMA (1.93 g, 9.9 mmol), MAETA (2.07 g, 9.9 mmol), and 2,2,2-trifluoroethyl methacrylate (6.00 g, 35.7 mmol) were dissolved in this order in dimethyl sulfoxide (DMSO, 40 mL). Azobisisobutyronitrile (AIBN, 0.01 g, Aldrich) was added to the flask. The flask was sealed with a rubber septum. Nitrogen was bubbled through the reaction mixture for 30 minutes to purge any dissolved oxygen. The flask was then kept at 60° C., while stirring at 300 rpm for 17 hours. After the reaction, 1 g of 4-methoxyphenol (MEHQ) was added to terminate the synthesis. The reaction mixture, which was observed to be viscous, was then precipitated in 1:1 acetone:hexane mixture by volume, purified by stirring three fresh portions of 1:1 acetone:hexane mixture by volume for at least 5 hours, one fresh portion of acetone for at least 5 hours, and three fresh portions of DI water for at least 5 hours. Finally, the terpolymer was dried in the vacuum oven for 48 h at 60° C. The composition of the terpolymer was calculated from the H-NMR spectrum (FIG. 13), using the ratio of protons from the three different monomer units. The calculated composition was: 15 mol % SEMA, 15 mol % MAETA, and 70 mol % TFEMA. The final conversion of this reaction was 40%.

### Example 13. Formation of Thin Film Composite Membranes from PTMS2

In this example, a membrane was prepared using the polymer described in example 12. The terpolymer (0.5 g) was dissolved in trifluoroethanol (TFE, 9.5 g) at approximately 25° C. The terpolymer solution was passed through a 0.45 micrometer syringe filter (Whatman) and degassed in a vacuum oven for at least 1 hours. The membranes were prepared by coating a thin layer of terpolymer solution on a commercial ultrafiltration (UF) membrane using a film applicator rod. PS35 ultrafiltration membrane, purchased from Solecta (Oceanside, Calif.), was used as the base membrane. After coating, the membrane was immersed in a water bath. Film thickness and morphology was determined by the examination of freeze-fractured cross-sections of the membranes using a scanning electron microscope (SEM, Phenom G2 Pure Tabletop SEM). In FIG. 14, SEM images of the uncoated base membrane (right) and the coated membrane (left) are given at the same magnification. The coating layer can be observed, it is dense (i.e. without macroscopic pores) with a thickness of about 0.7 micrometers.

### Example 14. Synthesis of Poly (2,2,2-trifluoroethyl methacrylate)-random-poly (2-(methacryloyloxy)ethyl]trimethylammonium chloride)-random-poly (2-sulfoethyl methacrylate) (PTMS3)

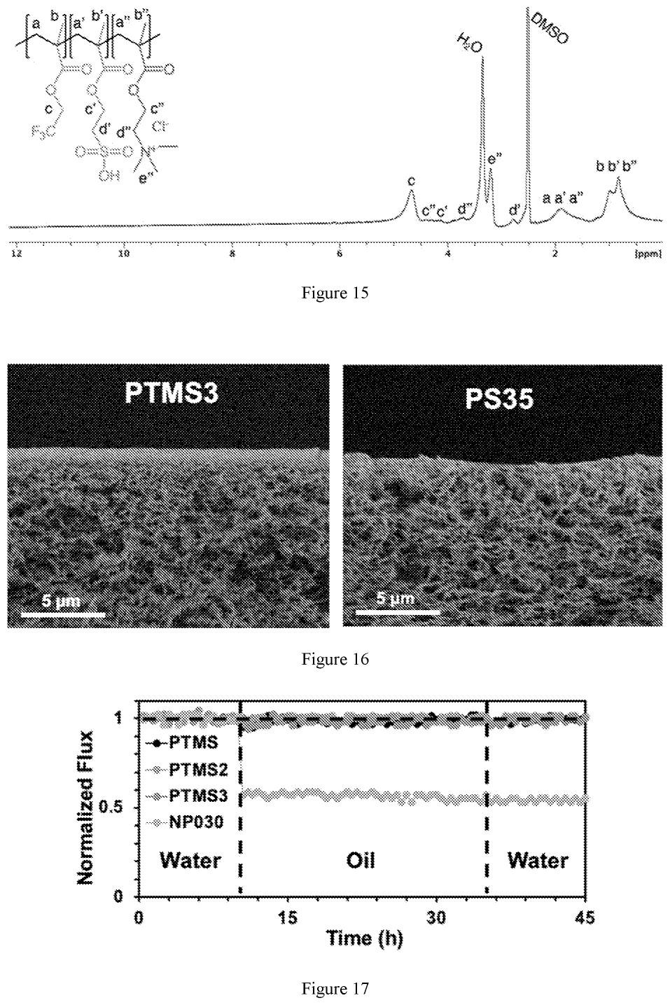

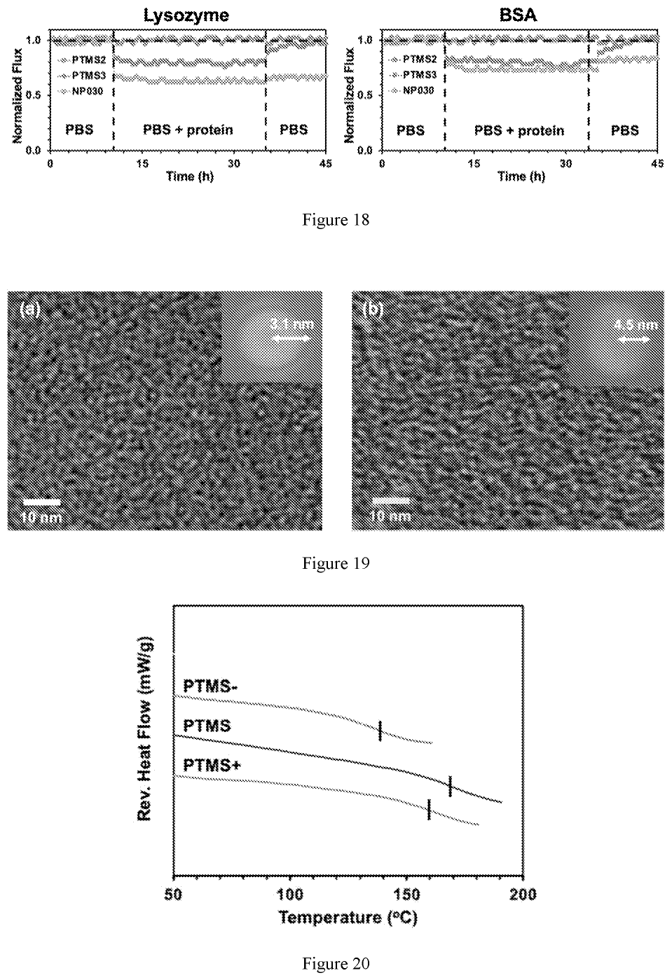

In this example, a random terpolymer with the three monomeric units being: 2-sulfoethyl methacrylate (SEMA, Polysciences), [2-(methacryloyloxy)ethyl]trimethylammonium chloride (MAETA, Sigma Aldrich), and 2,2,2-trifluoroethyl methacrylate (TFEMA, ACROS Organics). The first two monomers were passed through a column of neutral activated alumina (Sigma Aldrich) to remove the inhibitor, while the last monomer was passed through a column of basic activated alumina (Sigma Aldrich). SEMA (1.59 g, 8.2 mmol), MAETA (1.71 g, 8.2 mmol), and 2,2,2-trifluoroethyl methacrylate (6.70 g, 39.9 mmol) were dissolved in this order in dimethyl sulfoxide (DMSO, 40 mL). Azobisisobutyronitrile (AIBN, 0.01 g, Aldrich) was added to the flask. The flask was sealed with a rubber septum. Nitrogen was bubbled through the reaction mixture for 30 minutes to purge any dissolved oxygen. The flask was then kept at 60° C., while stirring at 300 rpm for 17 hours. After the reaction, 1 g of 4-methoxyphenol (MEHQ) was added to terminate the synthesis. The reaction mixture, which was observed to be viscous, was then precipitated in DI water, purified by stirring two fresh portions of DI water for at least 5 hours, and two fresh portions of isopropyl alcohol for at least 5 hours. Finally, the terpolymer was dried in the vacuum oven for 48 h at 60° C. The composition of the terpolymer was calculated from the ¹H-NMR spectrum (FIG. 15), using the ratio of protons from the three different monomer units. The calculated composition was: 11 mol % SEMA, 11 mol % MAETA, and 78 mol % TFEMA. The final conversion of this reaction was 70%.

### Example 15. Formation of Thin Film Composite Membranes from PTMS3

In this example, a membrane was prepared using the polymer described in example 14. The terpolymer (0.5 g) was dissolved in trifluoroethanol (TFE, 9.5 g) at approximately 25° C. The terpolymer solution was passed through a 0.45 micrometer syringe filter (Whatman) and degassed in a vacuum oven for at least 1 hours. The membranes were prepared by coating a thin layer of terpolymer solution on a commercial ultrafiltration (UF) membrane using a film applicator rod. PS35 ultrafiltration membrane, purchased from Solecta (Oceanside, Calif.), was used as the base membrane. After coating, the membrane was immersed in a water bath.

Film thickness and morphology was determined by the examination of freeze-fractured cross-sections of the membranes using a scanning electron microscope (SEM, Phenom G2 Pure Tabletop SEM). In FIG. 16, SEM images of the uncoated base membrane (right) and the coated membrane (left) are given at the same magnification. The coating layer can be observed, it is dense (i.e. without macroscopic pores) with a thickness of about 0.8 micrometers.

### Example 16. Water Permeance of Polyampholyte Terpolymer Membranes

In this example, the pure water fluxes through the membranes described in examples 2, 4, 6, 8, 10, 13, and 15 were measured using an Amicon 8010 stirred, dead-end filtration cell (Millipore) with a cell volume of 10 mL and an effective filtration area of 4.1 cm². The cell was stirred at 500 rpm, and the test was performed at 40 psi. After a stabilization period of at least one hour, a sample of the permeate was collected over 120 minutes and weighed. The value obtained was divided by filtration area and experiment time to obtain flux. The flux value was normalized by pressure to obtain pure water permeance (Table 1). These membranes have permeances comparable to nanofiltration and ultrafiltration commercial membranes.

TABLE 1
Water permeance of the seven terpolymer membranes.

| Terpolymer | Permeance (L/m² · h · bar) |
|---|---|
| PTMS | 2 |
| PTMS− | 5 |
| PTMS+ | 10 |
| PTMM | 28 |
| PTMT− | 18 |
| PTMS2 | 0.9 |
| PTMS3 | 0.4 |

### Example 17. Dye Rejection Using Polyampholyte Terpolymer Membranes

In this example, the membranes prepared as described in examples 2, 4, 6, 8, 10, 13, and 15 were used in experiments aimed at identifying their effective pore size, or size cut-off. We used dye molecules to probe this property, because dye molecules are rigid, and their concentrations are easily measured by UV-Vis spectroscopy. The retention experiments were performed on an Amicon 8010 stirred, dead-end filtration cell (Millipore) with a cell volume of 10 mL and an effective filtration area of 4.1 cm². The cell was stirred at 500 rpm, and the test was performed at 40 psi. The cell was stirred at 500 rpm to minimize concentration polarization effects. After running pure water through the membrane for at least an hour, the cell was emptied, and a 0.1 mM solution of the probe dye in water was placed in the cell. After rejecting the first 1 mL, to avoid pure water contamination, enough volume was collected for analysis by UV-Visible spectrophotometry and the solute rejection (%) was calculated (Table 2). The cell was rinsed several times with water. Pure water was filtered through the membrane until the permeate was completely clear before switching to a new solute.

TABLE 2
Rejection of neutral solute vitamin B12 (1,355
Da) by the seven terpolymer membranes.

| Terpolymer | Rejection (%) |
|---|---|
| PTMS | 98 |
| PTMS− | 89 |
| PTMS+ | 88 |
| PTMM | 57 |
| PTMT− | 44 |
| PTMS2 | 99 |
| PTMS3 | 98 |

### Example 18. Salt Rejection Using Polyampholyte Terpolymer Membranes

In this example, the membranes prepared as described in examples 2, 4, 6, 8, 10, 13, and 15 were used in experiments to determine their salt retention properties. We used different salts at different concentrations to probe this property, their concentrations were easily measured by a standard conductivity probe. The retention experiments were performed on an Amicon 8010 stirred, dead-end filtration cell (Millipore) with a cell volume of 10 mL and an effective filtration area of 4.1 cm². The cell was stirred at 500 rpm, and the test was performed at 40 psi. The cell was stirred at 500 rpm to minimize concentration polarization effects. After running pure water through the membrane for at least an hour, the cell was emptied, and a solution of the probe salt in water was placed in the cell. After rejecting the first 1 mL, to avoid pure water contamination, enough volume was collected for analysis by the conductivity probe and the salt rejection (%) was calculated (Table 3). The cell was rinsed several times with water. Pure water was filtered through the membrane until the permeate volume reaches 1 mL before switching to a new salt solution.

TABLE 3
Rejection of different salt solutions
by the seven terpolymer membranes.

| | Rejection (%) | | | | |
|---|---|---|---|---|---|
| | Na₂SO₄ | Na₂SO₄ | MgCl₂ | MgCl₂ | NaCl |
| Terpolymer | 1 mM | 10 mM | 1 mM | 10 mM | 1 mM |
| PTMS | 76 ± 1 | 76 ± 2 | 32 ± 2 | 30 ± 1 | 8 ± 1 |
| PTMS− | 96 ± 1 | 93 ± 2 | 18 ± 1 | 6 ± 1 | 54 ± 5 |
| PTMS+ | 17 ± 2 | 5 ± 1 | 83 ± 3 | 47 ± 5 | 23 ± 2 |
| PTMM | 12 ± 1 | 7 ± 1 | 10 ± 1 | 10 ± 1 | 4 ± 2 |
| PTMT− | 78 ± 2 | 58 ± 4 | 10 ± 1 | 7 ± 1 | 42 ± 4 |
| PTMS2 | 83 ± 2 | 84 ± 2 | 86 ± 2 | 87 ± 1 | 11 ± 2 |
| PTMS3 | 78 ± 2 | 79 ± 2 | 37 ± 3 | 36 ± 2 | 9 ± 1 |

### Example 19. Fouling Resistance of Polyampholyte Terpolymer Membranes

In this example, the membranes prepared as described in examples 2, 13, and 15 were used in experiments to determine their fouling resistance properties. The retention experiments were performed on an Amicon 8010 stirred, dead-end filtration cell (Millipore) with a cell volume of 10 mL and an effective filtration area of 4.1 cm². The cell was stirred at 500 rpm.

For the first test the feed solution consisted of a 1500 ppm oil-in-water emulsion (9:1 ratio of soybean oil:DC 193 surfactant obtained from Dow-Corning), prepared by blending the oil, water, and surfactant using a blender in high rpm for ˜3 min.

The second fouling test used a positively charged protein, lysozyme, the feed solution consisted of a 1 g/L lysozyme solution in 1× phosphate-buffered saline (PBS) buffer. While the third fouling test used a negatively charged protein, bovine serum albumin (BSA), the feed solution consisted of a 1 g/L BSA solution in 1× phosphate-buffered saline (PBS) buffer.

### Example 20. Self-Assembly of Polyampholyte Terpolymer Membranes

We document the self-assembled morphology of our r-PACs using transmission electron microscopy (TEM) images, and differential scanning calorimetry (DSC) analyses.

TEM was performed using a Hitachi 7800 transmission electron microscope operated in bright-field mode at 100 keV. To generate good contrast, ionic domains were preferentially stained by immersion in 2% aqueous CuCl₂ solution, resulting in the formation of sulfonate-copper complexes. FIG. 19 shows brightfield TEM images of PTMS and PTMS-copolymers. We observed a bicontinuous network of percolated ionic nanochannels (dark regions) surrounded by the hydrophobic phase (light regions) for both compositions. The fast Fourier transform (FFT) of the TEM image lacked directional features (inset of FIG. 19), indicating a disordered network. The characteristic length scale given by the outer ring of the FFT was 3.1 nm and 4.5 nm for PTMS and PTMS-respectively, corresponding to a dry state average ionic domain size of 1.55 nm and 2.25 nm for PTMS and PTMS-respectively.

To characterize copolymer phase separation we utilized DSC, where different glass transition temperatures can be related to different phase separated structures. DSC was performed utilizing a TA Q100 series calorimeter (TA Instruments) coupled with a N₂ purge and cooling systems. 3-5 mg of each polymer was sealed in aluminum pans, and fully dried under N₂ in the DSC chamber to avoid T_{g} shifts due to different water contents. After samples were fully dried a modulated heating ramp of 5° C./min was used. The T_{g} of all samples was obtained from the midpoint of the baseline shift.

### Example 21. Small Neutral Molecule Rejections Using Polyampholyte Terpolymer Membranes

In this example, the membranes prepared as described in examples 2, 4, 6, 8, 10, 13, and 15 were used in experiments aimed at identifying their effective pore size, or size cut-off. Membrane size-based selectivity was determined by filtering a series of small and neutral organic solutes at a 4,000 ppm concentration. The retention experiments were performed on an Amicon 8010 stirred, dead-end filtration cell (Millipore) with a cell volume of 10 mL and an effective filtration area of 4.1 cm². The cell was stirred at 500 rpm, and the test was performed at 40 psi. The cell was stirred at 500 rpm to minimize concentration polarization effects. After running pure water through the membrane for at least an hour, the cell was emptied, and a 4,000 ppm solution of the probe small molecule in water was placed in the cell. After rejecting the first 1 mL, to avoid pure water contamination, enough volume was collected for analysis by UV-Visible spectrophotometry utilizing COD kits (K-7365, CHEMetrics), in this manner solute rejection (%) was calculated (Table 4). The cell was rinsed several times with water. Pure water was filtered through the membrane until the permeate was completely clear before switching to a new solute.

TABLE 4
Rejection of different neutral solutes
by the five terpolymer membranes.

| | Rejection (%) | | |
|---|---|---|---|
| Terpolymer | Sucrose | Glucose | Glycerol |
| PTMS | 78 | 25 | 10 |
| PTMS− | 34 | 5 | 1 |
| PTMS+ | 36 | 7 | 1 |
| PTMS2 | 87 | 59 | 17 |
| PTMS3 | 76 | 23 | 9 |

### REFERENCES CITED

1. Ekblad, T.; Bergstroem, G.; Ederth, T.; Conlan, S. L.; Mutton, R.; Clare, A. S.; Wang, S.; Liu, Y. L.; Zhao, Q.; D'Souza, F.; Donnelly, G. T.; Willemsen, P. R.; Pettitt, M. E.; Callow, M. E.; Callow, J. A.; Liedberg, B., Poly(ethylene glycol)-Containing Hydrogel Surfaces for Antifouling Applications in Marine and Freshwater Environments. Biomacromolecules 2008, 9 (10), 2775-2783.
2. Grozea, C. M.; Walker, G. C., Approaches in designing non-toxic polymer surfaces to deter marine biofouling. Soft Matter 2009, 5 (21), 4088-4100.
3 Lejars, M.; Margaillan, A.; Bressy, C., Fouling release coatings: a nontoxic alternative to biocidal antifouling coatings. Chemical reviews 2012, 112 (8), 4347-4390.
4. Callow, J. A.; Callow, M. E., Trends in the development of environmentally friendly fouling-resistant marine coatings. Nature Communications 2011, 2, 244.
5. Wang, Z.; Hou, D.; Lin, S., Composite Membrane with Underwater-Oleophobic Surface for Anti-Oil-Fouling Membrane Distillation. Environmental Science & Technology 2016, 50 (7), 3866-3874.
6. Cremaschi, L.; Wu, X., Effect of fouling on the thermal performance of condensers and on the water consumption in cooling tower systems. Heat Transfer Engineering 2015, 36 (7-8), 663-675.
7. Charpentier, T.; Neville, A.; Baraka-Lokmane, S.; Hurtevent, C.; Ordonez-Varela, J.; Nielsen, F. M.; Eroini, V.; Olsen, J.; Ellingsen, J.; Bache, Ø. In Evaluation of anti-fouling surfaces for prevention of mineral scaling in sub-surface safety valves, SPE International Oilfield Scale Conference and Exhibition, Society of Petroleum Engineers: 2014.
8. Al-Janabi, A.; Malayeri, M.; Müller-Steinhagen, H., Experimental fouling investigation with electroless Ni—P coatings. International Journal of Thermal Sciences 2010, 49 (6), 1063-1071.
9. Kazi, S.; Duffy, G.; Chen, X., Fouling and fouling mitigation on heated metal surfaces. Desalination 2012, 288, 126-134.
10. Melo, L. F.; Bott, T. R., Biofouling in water systems. Experimental Thermal and Fluid Science 1997, 14 (4), 375-381.
11. Li, H.; Hsieh, M.-K.; Chien, S.-H.; Monnell, J. D.; Dzombak, D. A.; Vidic, R. D., Control of mineral scale deposition in cooling systems using secondary-treated municipal wastewater. Water research 2011, 45 (2), 748-760.
12. Rosmaninho, R.; Santos, O.; Nylander, T.; Paulsson, M.; Beuf, M.; Benezech, T.; Yiantsios, S.; Andritsos, N.; Karabelas, A.; Rizzo, G., Modified stainless steel surfaces targeted to reduce fouling-Evaluation of fouling by milk components. Journal of Food Engineering 2007, 80 (4), 1176-1187.
13. Guo, W. S.; Ngo, H. H.; Li, J. X., A mini-review on membrane fouling. Bioresource Technology 2012, 122, 27-34.
14. Flemming, H. C.; Schaule, G.; Griebe, T.; Schmitt, J.; Tamachkiarowa, A., Biofouling—the Achilles heel of membrane processes. Desalination 1997, 113, 215-225.
15. Paul, D., Reverse osmosis: scaling, fouling and chemical attack. Desal Water Reuse 1991, 1, 8-11.
16. Fane, A. G.; Fell, C. J. D., A review of fouling and fouling control in ultrafiltration. Desalination 1987, 62, 117-136.
17. Pervov, A. G., Scale Formation Prognosis and Cleaning Procedure Schedules in Reverse-Osmosis Systems Operation. Desalination 1991, 83 (1-3), 77-118.
18. Holmlin, R. E.; Chen, X. X.; Chapman, R. G.; Takayama, S.; Whitesides, G. M., Zwitterionic SAMs that resist nonspecific adsorption of protein from aqueous buffer. Langmuir 2001, 17 (9), 2841-2850.
19. Ostuni, E.; Chapman, R. G.; Holmlin, R. E.; Takayama, S.; Whitesides, G. M., A survey of structure-property relationships of surfaces that resist the adsorption of protein. Langmuir 2001, 17 (18), 5605-5620.
20. Luk, Y. Y.; Kato, M.; Mrksich, M., Self-assembled monolayers of alkanethiolates presenting mannitol groups are inert to protein adsorption and cell attachment. Langmuir 2000, 16 (24), 9604-9608.

21 Statz, A. R.; Barron, A. E.; Messersmith, P. B., Protein, cell and bacterial fouling resistance of polypeptoid-modified surfaces: effect of side-chain chemistry. Soft Matter 2008, 4 (1), 131-139.
22. Statz, A. R.; Meagher, R. J.; Barron, A. E.; Messersmith, P. B., New peptidomimetic polymers for antifouling surfaces. Journal of the American Chemical Society 2005, 127 (22), 7972-7973.
23. Zhang, Z.; Chao, T.; Chen, S. F.; Jiang, S. Y., Superlow fouling sulfobetaine and carboxybetaine polymers on glass slides. Langmuir 2006, 22 (24), 10072-10077.
24. Zhang, Z.; Chen, S. F.; Chang, Y.; Jiang, S. Y., Surface grafted sulfobetaine polymers via atom transfer radical polymerization as superlow fouling coatings. Journal of Physical Chemistry B 2006, 110 (22), 10799-10804.
25. Barcellona, M. N.; Johnson, N.; Bernards, M. T., Characterizing Drug Release from Nonfouling Polyampholyte Hydrogels. Langmuir 2015, 31 (49), 13402-9.
26. Zhang, W.; Yang, Z.; Kaufman, Y.; Bernstein, R., Surface and anti-fouling properties of a polyampholyte hydrogel grafted onto a polyethersulfone membrane. J. Colloid Interface Sci. 2018, 517, 155-165.
27. Yang, R.; Xu, J. J.; Ozaydin-Ince, G.; Wong, S. Y.; Gleason, K. K., Surface-Tethered Zwitterionic Ultrathin Antifouling Coatings on Reverse Osmosis Membranes by Initiated Chemical Vapor Deposition. Chemistry of Materials 2011, 23 (5), 1263-1272.
28. Zhai, G. Q.; Toh, S. C.; Tan, K. L.; Kang, E. T.; Neoh, K. G.; Huang, C. C.; Liaw, D. J., Poly(vinylidene fluoride) with grafted zwitterionic polymer side chains for electrolyte-responsive microfiltration membranes. Langmuir 2003, 19, 7030-7037.
29. Kitano, H.; Kawasaki, A.; Kawasaki, H.; Morokoshi, S., Resistance of zwitterionic telomers accumulated on metal surfaces against nonspecific adsorption of proteins. Journal of Colloid and Interface Science 2005, 282 (2), 340-348.
30. Sun, Q.; Su, Y. L.; Ma, X. L.; Wang, Y. Q.; Jiang, Z. Y., Improved antifouling property of zwitterionic ultrafiltration membrane composed of acrylonitrile and sulfobetaine copolymer. Journal of Membrane Science 2006, 285 (1-2), 299-305.
31. Kitano, H.; Imai, M.; Gemmei-Ide, M.; Takaha, K., Raman spectroscopic study on the structure of water in aqueous solution of zwitterionic surfactants. Journal of Colloid and Interface Science 2004, 269 (2), 459-465.
32. Straub, A. P.; Asa, E.; Zhang, W.; Nguyen, T. H.; Herzberg, M., In-situ graft-polymerization modification of commercial ultrafiltration membranes for long-term fouling resistance in a pilot-scale membrane bioreactor. Chem Eng J 2020, 382.
33. Bengani, P.; Kou, Y. M.; Asatekin, A., Zwitterionic copolymer self-assembly for fouling resistant, high flux membranes with size-based small molecule selectivity. Journal of Membrane Science 2015, 493, 755-765.
34. Bengani-Lutz, P.; Converse, E.; Cebe, P.; Asatekin, A., Self-Assembling Zwitterionic Copolymers as Membrane Selective Layers with Excellent Fouling Resistance: Effect of Zwitterion Chemistry. Acs Appl Mater Inter 2017, 9 (24), 20859-20872.
35. Bengani, P.; Kou, Y.; Asatekin, A., Zwitterionic copolymer self-assembly for fouling resistant, high flux membranes with size-based small molecule selectivity. Journal of Membrane Science 2015, 493, 755-765.
36. Bengani-Lutz, P.; Zaf, R. D.; Culfaz-Emecen, P. Z.; Asatekin, A., Extremely fouling resistant zwitterionic copolymer membranes with ˜1 nm pore size for treating municipal, oily and textile wastewater streams. Journal of Membrane Science 2017, 543 (Supplement C), 184-194.
37. Asatekin Alexiou, A.; Bengani, P. Zwitterion Containing Membranes. U.S. Pat. No. 10,150,088, 2018; incorporated by reference.
38. Lounder, S. J.; Asatekin, A., Zwitterionic Ion-Selective Membranes with Tunable Subnanometer Pores and Excellent Fouling Resistance. Chemistry of Materials 2021.
39. Lounder, S. J.; Asatekin Alexiou, A. Cross-linkable Zwitterionic Polymers and Their Use in Membrane Filters. application 63/025,559, filed May 15, 2020, pending; incorporated by reference.
40. Asatekin Alexiou, A.; Lounder, S. J. Charged Zwitterionic Copolymer Membranes. U.S. Patent Application 62/846,014, filed May 10, 2019; incorporated by reference.

### INCORPORATION BY REFERENCE

All U.S. patents, and U.S. and PCT patent application publications mentioned herein are hereby incorporated by reference. In case of conflict, the present application, including any definitions herein, will control.

### EQUIVALENTS

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

What is claimed is:

1. A polymer, comprising:
(a) a plurality of hydrophobic repeat units;
(b) a plurality of a cationic repeat units; and
(c) a plurality of an anionic repeat units;
wherein the hydrophobic repeat units, cationic repeat units, and anionic repeat units are in random or statistical order.

2. The polymer of claim 1, wherein the polymer is insoluble in water.

3. The polymer of claim 1 or 2, wherein the molar ratio of cationic repeat units to anionic repeat units is about 1.1:1 to about 1:1.1.

4. The polymer of any one of claims 1-3, wherein the molar ratio of cationic repeat units to anionic repeat units is about 1:1.

5. The polymer of any one of claims 1-4, wherein the net charge of the polymer is from −10 to +10.

6. The polymer of claim 1 or 2, wherein the molar ratio of cationic repeat units to anionic repeat units is greater than about 1:1, greater than about 1.5:1, or greater than about 2:1.

7. The polymer of claim 6, wherein the net charge of the polymer is positive.

8. The polymer of claim 1 or 2, wherein the molar ratio of cationic repeat units to anionic repeat units is less than about 1:1, less than about 0.67:1, or less than about 0.5:1.

9. The polymer of claim 8, wherein the net charge of the polymer is negative.

10. The polymer of any one of claims 1-9, wherein the molecular weight of the polymer is greater than about 20,000 g/mol, greater than about 40,000 g/mol, greater than about 60,000 g/mol, greater than about 80,000 g/mol, greater than about 100,000 g/mol, or greater than about 120,000 g/mol.

11. The polymer of claim 10, wherein the molecular weight of the polymer is greater than about 100,000 g/mol.

12. The polymer of any one of claims 1-11, wherein the plurality of hydrophilic repeat units constitutes at least about 30 wt %, at least about 35 wt %, at least about 40 wt %, at least about 45 wt %, at least about 50 wt %, at least about 60 wt %, at least about 65 wt %, at least about 70 wt %, at least about 75 wt %, at least about 80 wt %, at least about 85 wt %, or at least about 90 wt % of the polymer.

13. The polymer of any one of claims 1-11, wherein the plurality of hydrophilic repeat units constitutes about 30 wt % to about 99 wt %, about 30 wt % to about 90 wt %, about 40 wt % to about 90 wt %, about 40 wt % to about 80 wt %, about 40 wt % to about 75 wt %, about 40 wt % to about 70 wt %, about 45 wt % to about 75 wt %, about 45 wt % to about 70 wt %, about 50 wt % to about 80 wt %, about 50 wt % to about 75 wt %, or about 50 wt % to about 70 wt % of the polymer.

14. The polymer of claim 13, wherein the plurality of hydrophilic repeat units constitutes about 40 wt % to about 90 wt % of the polymer.

15. The polymer of claim 13, wherein the plurality of hydrophilic repeat units constitutes about 40 wt % to about 75 wt %, preferably about 50 wt % to about 70 wt % of the polymer.

16. The polymer of any one of claims 1-13, wherein the plurality of cationic repeat units and the plurality of anionic repeat units each constitutes at least about 0.5 wt %, at least about 1 wt %, at least about 2.5 wt %, at least about 5 wt %, at least about 10 wt %, at least about 15 wt %, at least about 20 wt %, at least about 25 wt %, at least about 30 wt %, at least about 35 wt %, or at least about 40 wt % of the polymer.

17. The polymer of any one of claims 1-16, wherein the plurality of cationic repeat units and the plurality of anionic repeat units each constitutes about 0.5 wt % to about 30 wt %, about 0.5 wt % to about 25 wt %, about 1 wt % to about 30 wt %, about 1 wt % to about 25 wt %, about 2 wt % to about 30 wt %, about 2 wt % to about 25 wt %, about 2.5 wt % to about 30 wt %, about 2.5 wt % to about 25 wt %, about 5 wt % to about 30 wt %, or about 5 wt % to about 25 wt % of the polymer.

18. The polymer of any one of claims 1-17, wherein each hydrophobic repeat unit is independently selected from the group consisting of 2,2-trifluoroethyl methacrylate (TFEMA), fluorinated acrylates, methacrylates, pentafluoropropyl methacrylate, heptafluorobutyl methacrylate, pentafluorophenyl methacrylate, styrene, methyl methacrylate, and acrylonitrile.

19. The polymer of any one of claims 1-18, wherein each hydrophobic repeat unit is the same, and is selected from the group consisting of 2,2-trifluoroethyl methacrylate (TFEMA), fluorinated acrylates, methacrylates, pentafluoropropyl methacrylate, heptafluorobutyl methacrylate, pentafluorophenyl methacrylate, styrene, methyl methacrylate, and acrylonitrile.

20. The polymer of claim 19, wherein the hydrophobic repeat unit is 2,2-trifluoroethyl methacrylate (TFEMA).

21. The polymer of any one of claims 1-20, wherein each anionic repeat unit is a methacrylate, acrylate, methacrylamide, acrylamide, styrene, or vinyl derivative comprising a carboxylic acid, sulfonate, phosphate, or other ionizable or charged group.

22. The polymer of any one of claims 1-20, wherein each anionic repeat unit is independently selected from the group consisting of methacrylic acid (MAA), 2-sulfoethyl methacrylate (SEMA), L-tryptophan-methacrylamide (L-Try-MA), D-tryptophan-methacrylamide, L or D-tryptophan-acrylamide, L or D-alanine-methacrylamide, L or D-alanine-acrylamide, L or D-valine-methacrylamide, L or D-valine-acrylamide, L or D-isoleucine-methacrylamide, L or D-isoleucine-acrylamide, L or D-allo-isoleucine-methacrylamide, L or D-allo-isoleucine-acrylamide, L or D-methionine-methacrylamide, L or D-methionine-acrylamide, L or D-phenylalanine-methacrylamide, L or D-phenylalanine-acrylamide, L or D-tyrosine-methacrylamide, L or D-tyrosine-acrylamide, L or D-histadine-methacrylamide, L or D-histadine-acrylamide, L or D-glutamic acid-methacrylamide, L or D-glutamic acid-methacrylamide, L or D-aspartic acid-acrylamide, L or D-aspartic acid-methacrylamide, acrylic acid, 2-carboxyethyl acrylate, mono-2-(methacryloyloxy)ethyl succinate, mono-2-(methacryloyloxy)ethyl maleate, sodium 4-vinylbenzenesulfonate, 2-acrylamido-2-methylpropane sulfonic acid, 2-acrylamido-2-methyl-1-propanesulfonic acid sodium salt, 2-acrylamido-2-methyl-1-propanesulfonic acid, 3-sulfopropyl acrylate potassium salt, 3-sulfopropyl methacrylate potassium salt, 3-vinylbenzoic acid, 4-vinylbenzoic acid, 2-vinylbenzoic acid, 4-(2-propenyl)benzoic acid, 2-methyl-2-propene-1-sulfonic acid sodium salt, vinylsulfonic acid sodium salt, vinylphosphonic acid, and (4-ethenylphenyl)methylphosphonic acid.

23. The polymer of any one of claims 1-20, wherein each anionic repeat unit is the same, and is selected from the group consisting of methacrylic acid (MAA), 2-sulfoethyl methacrylate (SEMA), L-tryptophan-methacrylamide (L-Try-MA), D-tryptophan-methacrylamide, L or D-tryptophan-acrylamide, L or D-alanine-methacrylamide, L or D-alanine-acrylamide, L or D-valine-methacrylamide, L or D-valine-acrylamide, L or D-isoleucine-methacrylamide, L or D-isoleucine-acrylamide, L or D-allo-isoleucine-methacrylamide, L or D-allo-isoleucine-acrylamide, L or D-methionine-methacrylamide, L or D-methionine-acrylamide, L or D-phenylalanine-methacrylamide, L or D-phenylalanine-acrylamide, L or D-tyrosine-methacrylamide, L or D-tyrosine-acrylamide, L or D-histadine-methacrylamide, L or D-histadine-acrylamide, L or D-glutamic acid-methacrylamide, L or D-glutamic acid-methacrylamide, L or D-aspartic acid-acrylamide, L or D-aspartic acid-methacrylamide, acrylic acid, 2-carboxyethyl acrylate, mono-2-(methacryloyloxy)ethyl succinate, mono-2-(methacryloyloxy)ethyl maleate, sodium 4-vinylbenzenesulfonate, 2-acrylamido-2-methylpropane sulfonic acid, 2-acrylamido-2-methyl-1-propanesulfonic acid sodium salt, 2-acrylamido-2-methyl-1-propanesulfonic acid, 3-sulfopropyl acrylate potassium salt, 3-sulfopropyl methacrylate potassium salt, 3-vinylbenzoic acid, 4-vinylbenzoic acid, 2-vinylbenzoic acid, 4-(2-propenyl)benzoic acid, 2-methyl-2-propene-1-sulfonic acid sodium salt, vinylsulfonic acid sodium salt, vinylphosphonic acid, and (4-ethenylphenyl)methylphosphonic acid.

24. The polymer of claim 23, wherein the anionic repeat unit is selected from the group consisting of methacrylic acid (MAA), 2-sulfoethyl methacrylate (SEMA), and L-tryptophan-methacrylamide (L-Try-MA).

25. The polymer of claim 23, wherein the anionic repeat unit is methacrylic acid (MAA).

26. The polymer of claim 23, wherein the anionic repeat unit is 2-sulfoethyl methacrylate (SEMA).

27. The polymer of any one of claims 1-26, wherein each cationic repeat unit is a methacrylate, acrylate, methacrylamide, acrylamide, styrene, or vinyl derivative comprising an amine, ammonium, pyridinium, imidazolium, phosphonium, pyrrolidinium, or other ionizable or charged group.

28. The polymer of any one of claims 1-26, wherein each cationic repeat unit is independently selected from the group consisting of [2-(methacryloyloxy)ethyl]trimethylammonium chloride (MAETA), [3-(methacryloylamino)propyl]trimethylammonium chloride, [2-(acryloyloxy)ethyl]trimethylammonium chloride, (3-acrylamidopropyl)trimethylammonium chloride, 4-vinylbenzyl(triphenyl)phosphonium chloride, (vinylbenzyl)trimethylammonium chloridemethacrylate, 3-vinylaniline, 4-vinylaniline, 2-isopropenylaniline, N-(3-aminopropyl)methacrylamide hydrochloride, N-vinylimidazolium salts, 1-allyl-3-vinylimidazolium salts, 1-allyl-2methyl-5-vinyl-pyridinium salt, 1-allyl-5-vinyl-pyridinium salt, and 1-methyl-1-(1-vinylcyclohexyl)pyrrolidinium iodide.

29. The polymer of any one of claims 1-26, wherein each cationic repeat unit is the same, and is selected from the group consisting of [2-(methacryloyloxy)ethyl]trimethylammonium chloride (MAETA), [3-(methacryloylamino)propyl]trimethylammonium chloride, [2-(acryloyloxy)ethyl]trimethylammonium chloride, (3-acrylamidopropyl)trimethylammonium chloride, 4-vinylbenzyl(triphenyl)phosphonium chloride, (vinylbenzyl)trimethylammonium chloridemethacrylate, 3-vinylaniline, 4-vinylaniline, 2-isopropenylaniline, N-(3-aminopropyl)methacrylamide hydrochloride, N-vinylimidazolium salts, 1-allyl-3-vinylimidazolium salts, 1-allyl-2methyl-5-vinyl-pyridinium salt, 1-allyl-5-vinyl-pyridinium salt, and 1-methyl-1-(1-vinylcyclohexyl)pyrrolidinium iodide.

30. The polymer of claim 29, wherein the cationic repeat unit is 2-(methacryloyloxy)ethyl]trimethylammonium chloride (MAETA).

31. The polymer of any one of claims 1-30, wherein the cationic repeat unit comprises a quaternary ammonium group.

32. The polymer of claim 1, wherein the polymer consists essentially of or consists of:
(a) a plurality of hydrophobic repeat units;
(b) a plurality of a cationic repeat units; and
(c) a plurality of an anionic repeat units;
wherein the hydrophobic repeat units, cationic repeat units, and anionic repeat units are in random or statistical order.

33. A thin film composite membrane, comprising a porous substrate, and a selective layer comprising a polymer of any one of claims 1-32, wherein an average effective pore size of the porous substrate is larger than an average effective pore size of the selective layer; and the selective layer is disposed on a surface of the porous substrate.

34. The thin film composite membrane of claim 33, wherein the selective layer has a thickness of about 10 nm to about 10 μm.

35. The thin film composite membrane of claim 34, wherein the selective layer has the thickness of about 10 nm to about 3 μm.

36. The thin film composite membrane of claim 35, wherein the selective layer has the thickness of about 10 nm to about 1 μm.

37. The thin film composite membrane of any one of claims 33-36, wherein the selective layer has the average effective pore size of about 0.1 nm to about 2.0 nm.

38. The thin film composite membrane of any one of claims 33-36, wherein the selective layer has the average effective pore size of about 0.1 nm to about 1.2 nm.

39. The thin film composite membrane of any one of claims 33-36, wherein the selective layer has the average effective pore size of about 0.7 nm to about 1.2 nm.

40. The thin film composite membrane of any one of claims 33-39, wherein the net charge of the polymer is positive, and the thin film composite membrane rejects positively charged solutes and salts.

41. The thin film composite membrane of any one of claims 33-39, wherein the net charge of the polymer is negative, and the thin film composite membrane rejects negatively charged solutes and salts.

42. The thin film composite membrane of any one of claims 33-39, wherein the net charge of the polymer is from −10 to +10; and the selective layer exhibits size-based selectivity between uncharged organic molecules.

43. The thin film composite membrane of claim 42, wherein the selective layer exhibits rejection of greater than or equal to about 95% or greater than or equal to about 99% for neutral molecule with hydrated diameter of about or greater than 1.5 nm.

44. The thin film composite membrane of any one of claims 33-43, wherein the selective layer exhibits antifouling properties.

45. The thin film composite membrane of any one of claims 33-43, wherein the selective layer exhibits resistance to fouling by an oil emulsion.

46. The thin film composite membrane of any one of claims 33-43, wherein the selective layer exhibits resistance to fouling by adsorption of bioorganic macromolecules (e.g., proteins, alginate, organic matter).

47. A material with an anti-fouling coating, comprising a solid substrate, and a layer comprising a polymer of any one of claims 1-32, wherein the layer is disposed on a surface of the solid substrate.

48. The anti-fouling coating of claim 47, wherein the layer has a thickness of about 10 nm to about 10 μm.

49. The anti-fouling coating of claim 47, wherein the layer has the thickness of about 10 nm to about 3 μm.

50. The anti-fouling coating of claim 47, wherein the layer has the thickness of about 10 nm to about 1 μm.

51. A method of purifying a peptide or protein, comprising:
contacting at a first flow rate the thin film composite membrane of any one of claims 33-46 with a mixture comprising one or more peptides or proteins, whereby a portion of the mixture is retained by the thin film composite membrane.

52. A method of removing an organic solute from water, comprising:
contacting at a first flow rate the thin film composite membrane of any one of claims 33-46 with an aqueous solution comprising an organic solute, whereby the organic solute is retained by the thin film composite membrane.

53. A method of decontaminating water, comprising:
contacting at a first flow rate the thin film composite membrane of any one of claims 33-46 with an mixture comprising water and one or more contaminants, whereby one or more contaminants are retained by the thin film composite membrane.

54. A method of size-selective separation, comprising:
contacting at a first flow rate the thin film composite membrane of any one of claims 33-46 with a mixture comprising one or more particles of differing sizes, whereby one or more particles of a particular size are retained by the thin film composite membrane.
